(19) United States
(12) Patent Application Publication
(10) Pub. No.: US 2025/0257322 A1
(43) Pub. Date: Aug. 14, 2025

(54) MODIFIED HEMATOPOIETIC STEM CELLS AND USES THEREOF

(71) Applicant: STEMCELL TECHNOLOGIES CANADA INC., VANCOUVER (CA)

(72) Inventors: Murillo SILVA, Watertown, MA (US); Devin BRIDGEN, Watertown, MA (US); Jonathan GILBERT, Watertown, MA (US)

(21) Appl. No.: 18/726,305

(22) PCT Filed: Jan. 4, 2023
(86) PCT No.: PCT/US2023/060116

§ 371 Date: Jul. 2, 2024

Related U.S. Application Data
(60) Provisional application No. 63/296,544, filed on Jan. 5, 2022.
(60) Provisional application No. 63/374,622, filed on Sep. 6, 2022.

(51) Int. Cl. C12N 5/0789; A61K 35/12; A61K 35/28; A61K 38/00; A61P 7/00; C07K 14/715

(52) U.S. Cl. CPC C12N 5/0647; A61K 35/28; A61P 7/00; C07K 14/7158; A61K 2035/124; A61K 38/00; C12N 2501/50; C12N 2510/00; C12N 2527/00

(57) ABSTRACT

The present disclosure provides methods for producing modified HSCs, wherein the method comprises passing a cell suspension comprising the cell and a payload through a constriction, wherein the constriction deforms the cell, thereby causing a perturbation of the cell such that the payload enters the cell. In some aspects, the payloads are capable of enhancing one or more properties of the HSCs, such that the HSCs are better engrafted within the bone marrow of a subject.

### CROSS-REFERENCE TO RELATED APPLICATIONS

This PCT application claims the priority benefit of U.S. Provisional Application Nos. 63/296,544, filed Jan. 5, 2022, and 63/374,622, filed Sep. 6, 2022, each of which is herein incorporated by reference in its entirety.

### REFERENCE TO SEQUENCE LISTING SUBMITTED ELECTRONICALLY

The content of the electronically submitted sequence listing in .XML file (Name: 4821_007PC02_Seqlisting_ST26; Size: 20,966 bytes; and Date of Creation: Jan. 3, 2023) filed with the application is herein incorporated by reference in its entirety.

### FIELD OF DISCLOSURE

The present disclosure relates generally to methods of producing hematopoietic stem cells (HSCs) with enhanced properties (e.g., increased resistance to mobilization and/or apoptotic factors).

### BACKGROUND OF DISCLOSURE

Hemoglobinopathies are group of inherited monogenic blood cell disorders (e.g., Sickle Cell Disease and Thalassemia Syndrome) characterized by abnormal production or structure of the hemoglobin molecules. Modell et al., Bull World Health Organ 86:480-487 (2008); and Kohne et al., Dtsch Arztebl Int 108:532 (2011). Such disorders can be associated with severe pain and multi-organ ischemic damage, increased risk of infection, and even premature death. Current treatment options include small molecule-based therapies like hydroxyurea, L-glutamine and voxelotor, all of which exhibit limited efficacy and are non-curative. And, while hematopoietic stem cell transplantation (HSCT) has had some success, such a treatment regimen require the use of toxic myeloablative therapies to deplete the endogenous stem cell compartment to make space for the engraftment of therapeutically modified HSCs in the bone marrow niche. Busulfan, a commonly used myeloablative agent, has been associated with severe short-term and long-term clinical adverse events (e.g., cerebellar hemorrhage, myelodysplastic syndrome, veno-occlusive disease). Morales-Ramirez et al., Arch Med Res 37:316-321 (2006); Iwamoto et al., Cancer Sci 95: 454-458 (2004); and Finazii et al., Br J Haematol 110: 577-583 (2000). Accordingly, there remains a need for a more efficient and safer approach to HSC-based treatment regimens for the treatment of hemoglobinopathies and other similar disorders.

### SUMMARY OF DISCLOSURE

Disclosed herein is a method of producing a modified hematopoietic stem cell (HSC), comprising passing a cell suspension, which comprises a population of HSC, through a constriction under one or more parameters, wherein passing the cell suspension through the constriction under the one or more parameters allows a payload to enter the HSC, and wherein the payload is capable of modifying the HSC, such that the HSC exhibits: (i) increased resistance to a mobilization factor, (ii) increased resistance to an apoptotic factor, (iii) increased resistance to a depleting factor, (iv) increased expression of a homing factor, or (v) a combination thereof.

In some aspects, after the modifying, the resistance of the HSC to the mobilization factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, after the modifying, the resistance of the HSC to the apoptotic factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, after the modifying, the resistance of the HSC to the depleting factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, the expression of a homing factor on the HSC is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters.

Also provided herein is a method of increasing the homing of a hematopoietic stem cell (HSC) to the bone marrow of a subject in need thereof, comprising passing a cell suspension, which comprises the HSC, through a constriction under one or more parameters, wherein passing the cell suspension through the constriction under the one or more parameters allows a payload to enter the HSC, and wherein the payload is capable of modifying the HSC, such that the HSC exhibits increased homing to the bone marrow when administered to the subject.

In some aspects, after the modifying, the HSC exhibits increased expression of a homing receptor. In some aspects, the expression of the homing receptor on the HSC is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters.

The present disclosure further provides a method of increasing the survival of a hematopoietic stem cell in a subject in need thereof, comprising passing a cell suspension, which comprises the HSC, through a constriction under one or more parameters, wherein passing the cell suspension through the constriction under the one or more parameters allows a payload to enter the HSC, and wherein the payload is capable of modifying the HSC, such that the HSC exhibits increased survival when administered to the subject.

In some aspects, after the modifying, the HSC exhibits increased resistance to an apoptotic factor. In some aspects, the resistance of the HSC to the apoptotic factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters.

Provided herein is a method of promoting the engraftment of a hematopoietic stem cell (HSC) in the bone marrow of a subject in need thereof, comprising passing a cell suspension, which comprises the HSC, through a constriction under one or more parameters, wherein passing the cell suspension through the constriction under the one or more parameters allows a payload to enter the HSC, and wherein the payload is capable of modifying the HSC, such that the HSC is engrafted in the bone marrow of the subject when administered to the subject.

In some aspects, after the modifying, the HSC exhibits: (i) increased resistance to a mobilization factor, (ii) increased resistance to an apoptotic factor, (iii) increased resistance to a depleting factor, (iv) increased expression of a homing factor, or (v) a combination thereof.

In some aspects, the resistance of the HSC to the mobilization factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, the resistance of the HSC to the apoptotic factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, the resistance of the HSC to the depletion factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters. In some aspects, the expression of the homing factor on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to a corresponding HSC which was not passed through the constriction under the one or more parameters.

Also provided herein is a method of producing a modified hematopoietic stem cell (HSC), comprising intracellularly delivering a payload into a HSC, wherein the payload is capable of modifying the HSC, such that the HSC exhibits: (i) increased resistance to a mobilization factor, (ii) increased resistance to an apoptotic factor, (iii) increased resistance to a depleting factor, (iv) increased expression of a homing factor, or (v) a combination thereof. Also provided herein is a method of increasing the homing of a hematopoietic stem cell (HSC) to the bone marrow of a subject in need thereof, comprising intracellularly delivering a payload into a HSC, wherein the payload is capable of modifying the HSC, such that the HSC exhibits increased homing to the bone marrow when administered to the subject. Also provided herein is a method of increasing the survival of a hematopoietic stem cell (HSC) in a subject in need thereof, comprising intracellularly delivering a payload into a HSC, wherein the payload is capable of modifying the HSC, such that the HSC exhibits increased survival when administered to the subject. Also provided herein is a method of promoting the engraftment of a hematopoietic stem cell (HSC) in the bone marrow of a subject in need thereof, comprising intracellularly delivering a payloaod into a HSC, wherein the payload is capable of modifying the HSC, such that the HSC is engrafted in the bone marrow of the subject when administered to the subject. In some aspects, the payload is transiently expressed in the modified HSC.

In some aspects, the payload comprises a homing receptor, a cytokine, a growth factor, a cell adhesion molecule, a proliferative agent, a survival factor, a combination thereof, or a regulator thereof.

In some aspects, the homing receptor comprises a CXCR4, CXCR2, or both. In some aspects, the CXCR4 comprises an amino acid sequence which differs from the corresponding wild-type amino acid sequence set forth in SEQ ID NO: 1. In some aspects, the amino acid sequence of the CXCR4 comprises one of the following mutations: R334X, A175F, H113A, D171N, D262N, I284A, H281A, Q200W, Q200A, or a combination thereof.

In some aspects, the cytokine comprises a stem cell factor (SCF), a Fms-related tyrosine kinase 3 ligand (Flt3L), or both. In some aspects, the growth factor comprises a thrombopoietin (TPO). In some aspects, the cell adhesion molecule comprises an integrin, a selectin, or both. In some aspects, the cell adhesion molecule comprises VLA-4, VLA-5, LFA-1, or a combination thereof. In some aspects, the proliferative agent comprises an activator of a signaling pathway involved in cell proliferation. In some aspects, the signaling pathway comprises PI3K-ATK, Ras-ERK, or both. In some aspects, the survival factor comprises a Bcl-2, a Bcl-xL, a MCL-1, a Ced-9, a bfl-1, or a combination thereof.

In some aspects, the Bcl-2 comprises an amino acid sequence which differs from the corresponding wild-type amino acid sequence set forth in SEQ ID NO: 9. In some aspects, the amino acid sequence of the Bcl-2 comprises the G101V mutation, the D103Y mutation, or both.

In some aspects, the regulator is capable of increasing the expression and/or activity of the homing receptor, the cytokine, the growth factor, the cell adhesion molecule, the proliferative agent, the survival factor, or a combination thereof. In some aspects, the regulator comprises a prostaglandin (e.g., prostaglandin E₂(PGE2)). In some aspects, the regulator is capable of reducing or preventing (e.g., knocking down) the activity of an inhibitor of the homing receptor, the cytokine, the growth factor, the cell adhesion molecule, the proliferative agent, the survival factor, or a combination thereof.

In some aspects, the inhibitor comprises a GPRASP1/2, CD26, or both.

In some aspects, the mobilization factor comprises Plerixaflor, AMD3465, GROβ, G-CSF, anti-CD117 antibody, or a combination thereof. In some aspects, the apoptotic factor comprises a Bcl-2 inhibitor (e.g., Venetoclax), a MCL-1 inhibitor (e.g., S63845 or S64315), a BCL-XL inhibitor, or a combination thereof.

In some aspects, the payload comprises a nucleic acid. In some aspects, the nucleic acid comprises a DNA, a RNA, or both. In some aspects, the RNA comprises a mRNA, a siRNA, a miRNA, a lncRNA, a tRNA, a shRNA, a self-amplifying mRNA (saRNA), a PNA, a locked nucleic acid (LNA), or a combination thereof.

In some aspects, the above methods comprise contacting the HSC with the payload: (i) prior to the passing of the cell suspension through the constriction, (ii) during the passing of the cell suspension through the constriction, (iii) after the passing of the cell suspension through the constriction, or (iv) a combination thereof. In some aspects, the HSC is contacted with the payload prior to the passing of the cell suspension through the constriction. In some aspects, the HSC is contacted with the payload during the passing of the cell suspension through the constriction. In some aspects, the HSC is contacted with the payload after the passing of the cell suspension through the constriction.

In some aspects, the HSC is CD34+, CD90+, CD45RA−, and lineage marker negative (e.g., CD2, CD3, CD11b, CD11c, CD14, CD16, CD19, CD24, CD56, CD66b, and CD235).

In some aspects, the constriction is within a microfluidic chip.

In some aspects, the one or more parameters are selected from a cell density; pressure; length, width, and/or depth of the constriction; diameter of the constriction; diameter of the cells; temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or a combination thereof.

In some aspects, the cell density is at least about 1×10⁵ cells/mL, at least about 2×10⁵ cells/mL, at least about 3×10⁵ cells/mL, at least about 4×10⁵ cells/mL, at least about 5×10⁵ cells/mL, at least about 6×10⁵ cells/mL, at least about 7×10⁵ cells/mL, at least about 8×10⁵ cells/mL, at least about 9×10⁵ cells/mL, at least about 1×10⁶ cells/mL, at least about 2×10⁶ cells/mL, at least about 3×10⁶ cells/mL, at least about 4×10⁶ cells/mL, at least about 5×10⁶ cells/mL, at least about 6×10⁶ cells/mL, at least about 7×10⁶ cells/mL, at least about 8×10⁶ cells/mL, at least about 9×10⁶ cells/mL, at least about 1×10⁷ cells/mL, at least about 2×10⁷ cells/mL, at least about 3×10⁷ cells/mL, at least about 4×10⁷ cells/mL, at least about 5×10⁷ cells/mL, at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more.

In some aspects, the pressure is at least about 20 psi, at least about 25 psi, at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi.

In some aspects, the diameter of the constriction is about 20%, about 30%, about 40%, about 50%, about 60%, about 70%, about 80%, about 90%, or about 99% of the diameter of the HSC.

In some aspects, the length of the constriction is up to about 100 μm. In some aspects, the length of the constriction is less than about 0.1 μm, less than about 0.2 μm, less than about 0.3μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the length of the constriction is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width of the constriction is between about 1 μm to about 10 μm. In some aspects, the width of the constriction is about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 3 μm, about 4 μm, about 5 μm, about 6 μm, about 7 μm, about 8 μm, about 9 μm, or about 10 μm.

In some aspects, the depth of the constriction is at least about 1 μm. In some aspects, the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth of the constriction is about 5 μm to about 90 μm. In some aspects, the depth is about 5 μm, about 10 μm, about 20 μm, about 30 μm, about 40 μm, about 50 μm, about 60 μm, about 70 μm, about 80 μm, or about 90 μm.

In some aspects, the constriction comprises a width and a depth, wherein the width of the constriction is about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 3 μm, about 4 μm, about 5 μm, about 6 μm, about 7 μm, about 8 μm, about 9 μm, or about 10 μm, and wherein the depth of the constriction is about 5 μm, about 10 μm, about 20 μm, about 30 μm, about 40 μm, about 50 μm, about 60 μm, about 70 μm, about 80 μm, or about 90 μm.

In some aspects, the HSC is contacted with multiple payloads (i) prior to the passing of the cell suspension through the constriction, (ii) during the passing of the cell suspension through the constriction, (iii) after the passing of the cell suspension through the constriction, or (iv) a combination thereof, such that passing the cell suspension through the constriction under the one or more parameters allow at least two or more of the multiple payloads to enter the HSC.

In some aspects, the multiple payloads comprise at least about 2, at least about 3, at least about 4, at least about 5, at least about 6, at least about 7, at least about 8, at least about 9, or at least about 10 or more payloads. In some aspects, the at least two or more of the multiple payloads enter the cell concurrently. In some aspects, the at least two or more of the multiple payloads enter the cell sequentially.

In some aspects, the methods provided herein comprise passing the cell suspension through a plurality of constrictions. In some aspects, the plurality of constrictions comprise at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions.

In some aspects, each constriction of the plurality of constrictions are the same. In some aspects, one or more of the constrictions of the plurality of constrictions are different.

In some aspects, one or more of the constrictions differ in their length, depth, width, or combinations thereof. In some aspects, each constriction of the plurality of constrictions is associated with the same payload. In some aspects, one or more of the plurality of constrictions is associated with a different payload.

In some aspects, the plurality of constrictions are contained within a single microfluidic chip. In some aspects, the plurality of constrictions are contained within multiple microfluidic chips, wherein each of the multiple microfluidic chips comprises a constriction. In some aspects, each of the multiple microfluidic chips are the same. In some aspects, one or more of the multiple microfluidic chips are different.

In some aspects, the time interval between passing the cell suspension through a first constriction and a second constriction of the plurality of constrictions is less than about 1 μs, less than about 1 second, less than about 1 minute, less than about 30 minutes, less than about 1 hour, less than about 6 hours, less than about 12 hours, less than about 1 day, less than about 2 days, less than about 3 days, less than about 4 days, or less than about 5 days.

In some aspects, the plurality of constrictions comprise a first constriction associated with a first payload and a second constriction associated with a payload, wherein the cell suspension is passed through the first constriction allowing the first payload to enter the HSC, and then the cell suspension is passed through the second constriction allowing the second payload to enter the HSC.

Provided herein is a population of hematopoietic stem cells (HSCs) produced using any of the methods disclosed herein. Also provided herein is a composition comprising the population of HSCs produced herein, and a pharmaceutically acceptable carrier. Further provided herein is a kit comprising the population of HSCs produced herein, and instructions for use.

Provided herein is a composition comprising a population of modified hematopoietic stem cells (HSCs), wherein the modified HSCs comprise a payload, which is capable of: (i) increasing the resistance of the modified HSCs to a mobilization factor, (ii) increasing the resistance of the modified HSCs to an inhibitor of an anti-apoptotic factor, (iii) increasing the resistance of the modified HSCs to a depleting factor, (iv) increasing the expression of a homing factor on the modified HSCs, or (v) a combination thereof.

In some aspects, the resistance of the modified HSCs to a mobilization factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to corresponding HSCs which have not been modified to comprise the payload. In some aspects, the resistance of the modified HSCs to an inhibitor of an anti-apoptotic factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to corresponding HSCs which have not been modified to comprise the payload. In some aspects, the resistance of the modified HSCs to a depleting factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to corresponding HSCs which have not been modified to comprise the payload. In some aspects, the expression of a homing receptor on the modified HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to corresponding HSCs which have not been modified to comprise the payload.

In some aspects, the payload is transiently expressed in the modified HSCs. In some aspects, the payload comprises a homing receptor, a cytokine, a growth factor, a cell adhesion molecule, a proliferative agent, a survival factor, a combination thereof, or a regulator thereof. In some aspects, the homing receptor comprises a CXCR4, CXCR2, or both. In some aspects, the CXCR4 comprises an amino acid sequence which differs from the corresponding wild-type amino acid sequence set forth in SEQ ID NO: 1. In some aspects, the amino acid sequence of the CXCR4comprises one of the following mutations: R334X, A175F, H113A, D171N, D262N, 1284A, H281A, Q200W, Q200A, or a combination thereof. In some aspects, the cytokine comprises a stem cell factor (SCF), a Fms-related tyrosine kinase 3 ligand (Flt3L), or both. In some aspects, the growth factor comprises a thrombopoietin (TPO). In some aspects, the cell adhesion molecule comprises an integrin, a selectin, or both. In some aspects, the cell adhesion molecule comprises VLA-4, VLA-5, LFA-1, or a combination thereof. In some aspects, the proliferative agent comprises an activator of a signaling pathway involved in cell proliferation. In some aspects, the signaling pathway comprises PI3K-ATK, Ras-ERK, or both. In some aspects, the survival factor comprises a Bcl-2, a Bcl-XL, a MCL-1, a Ced-9, a bfl-1, or a combination thereof. In some aspects, the Bcl-2 comprises an amino acid sequence which differs from the corresponding wild-type amino acid sequence set forth in SEQ ID NO: 9. In some aspects, the amino acid sequence of the Bcl-2 comprises the G101V mutation, the D103Y mutation, or both. In some aspects, the regulator is capable of increasing the expression and/or activity of the homing receptor, the cytokine, the growth factor, the cell adhesion molecule, the proliferative agent, the survival factor, or a combination thereof. In some aspects, the regulator comprises a prostaglandin (e.g., prostaglandin E₂ (PGE2)). In some aspects, the regulator is capable of reducing or preventing (e.g., knocking down) the activity of an inhibitor of the homing receptor, the cytokine, the growth factor, the cell adhesion molecule, the proliferative agent, the survival factor, or a combination thereof. In some aspects, the inhibitor comprises a GPRASP1/2, CD26, or both. In some aspects, the mobilization factor comprises Plerixaflor, AMD3465, GROβ, G-CSF, anti-CD117 antibody, or a combination thereof. In some aspects, the apoptotic factor comprises a Bcl-2 inhibitor (e.g., Venetoclax), a MCL-1 inhibitor (e.g., S63845 or S64315), a BCL-XL inhibitor, or a combination thereof. In some aspects, the payload comprises a nucleic acid. In some aspects, the nucleic acid comprises a DNA, a RNA, or both. In some aspects, the RNA comprises a mRNA, a siRNA, a miRNA, a lncRNA, a tRNA, a shRNA, a self-amplifying mRNA (saRNA), a PNA, a locked nucleic acid (LNA), or a combination thereof.

Disclosed herein is a method of treating a disease or disorder in a subject in need thereof, comprising administering to the subject any of the population of HSCs produced herein or any of the compositios descrbed herein.

In some aspects, the disease or disorder comprises a blood disorder. In some aspects, the blood disorder comprises Sickle Cell Disease (SCD), Thalassemia Syndrome, severe aplastic anemia, Fanconi anemia, Paroxysmal nocturnal hemoglobinuria, Pure red cell aplasia, congenital amegakaryocytic thrombocytopenia, or a combination thereof.

In some aspects, the disease or disorder comprises an immune disorder. In some aspects, the immune disorder comprises Severe combined immunodeficiency, Wiskott-Aldrich syndrome, or both.

In some aspects, the disease or disorder comprises a metabolic disorder. In some aspects, the metabolic disorder comprises Krabbe disease (GLD), Hurler syndrome, Adrenoleukodystrophy, Metachromatic leukodystrophy, Fabry disease, Gaucher disease, cystinosis, Hunter syndrome, Pompe disease, or a combination thereof.

In some aspects, the subject does not receive a myeloablative preconditioning regimen prior to the administration of the population of HSCs or the composition. In some aspects, the myeloablative preconditioning regimen comprises an irradiation, a chemotherapy, a small molecule, an antibody, or a combination thereof.

### BRIEF DESCRIPTION OF FIGURES

FIG. 1 shows the engraftment of the squeeze processed human CD34+ HSCs in the bone marrow of NSG (top) and NBSGW (bottom) mice (not preconditioned) at six weeks post adoptive transfer. The engraftment of the HSCs is shown as a percentage of the total CD45+ bone marrow cells as measured using flow cytometry. “hCD45”=human CD45 (i.e., adoptively transferred human HSCs); and “mCD45”=mouse CD45 (i.e., endogenous mouse HSCs).

FIGS. 2A, 2B, and 2C show the frequency of the adoptively transferred human HSCs present within the peripheral blood (FIG. 2A), spleen (FIG. 2B), and the bone marrow (FIG. 2C) in NSG and NBSGW mice at six weeks post transfer. The frequency of the cells is shown as a percentage of the total CD45+ cells. “NBSGW NC”=squeeze processed HSCs without any payload and administered into NBSGW mice; “NBSGW SQZ”=squeezed processed HSCs with GFP mRNA and administered into NBSGW mice; “NSG NC”=squeeze processed HSCs without any payload and administered into NSG mice; and “NSG SQZ”=squeeze processed HSCs with GFP mRNA and administered into NSG mice—i.e., Groups A, B, C, and D, respectively, in Table 5 (see Example 1).

FIGS. 3A and 3B show the expression kinetics of different CXCR4 variants in human CD34+ HSCs squeeze processed with CXCR4 variant mRNA. The CXCR4 expression is shown at 4 hours, 24 hours, 48 hours, and 72 hours after the squeeze processing, as measured using flow cytometry. In FIG. 3A, the CXCR4 expression is shown as the geometric mean fluorescence intensity. In FIG. 3B, the CXCR4 variant expression is shown as a fold increase over the corresponding expression in the control HSCs (i.e., no payload and no squeeze processing). The different groups shown are as follows: (1) no payload and no squeeze processing “NC”; (2) squeeze processing without any payload (“empty”); (3) squeeze processing with wild-type CXCR4 mRNA (“CXCR4 wt”); (4) squeeze processing with CXCR4-A175F variant mRNA (“CXCR4 A175F”); (5) squeeze processing with CXCR4-A175F+R334X variant mRNA (“CXCR4 A175F R334X”); and (6) squeeze processing with CXCR4-R334X variant mRNA (“CXCR4 R334X”)—i.e., Groups A-F, respectively, in Table 6 (see Example 2).

FIG. 3C provide a comparison of CXCL12 signaling inhibition caused by plerixafor in human CD34+ HSCs squeeze processed with the wild-type CXCR4 mRNA (middle bar) or with the CXCR4 variant (comprising A175F modification) mRNA (right bar). Non-squeeze processed HSCs (i.e., treated with plerixafor alone) were used as control (left bar).

FIGS. 3D and 3E show CXCR4 expression in squeeze processed human CD34+ HSCs after adoptive transfer into NSG mice. FIG. 3D provides a schematic of the experimental design. As shown, prior to the transfer, the human CD34+ HSCs were squeeze processed with no payload (i.e., empty squeeze; “ES”) or mRNA encoding CXCR4 (i.e., a bone marrow homing receptor). FIG. 3E provides a comparison of the CXCR4 expression (geometric mean fluorescence intensity) by the adoptively transferred cells within the bone marrow at 24 hours post adoptive transfer.

FIGS. 4A and 4B show the expression kinetics of Bcl-2 variants in human CD34+ HSCs squeeze processed with Bcl-2 variant mRNA. The Bcl-2 expression is shown at 4 hours, 24 hours, 48 hours, and 72 hours after the squeeze processing, as measured using flow cytometry. In FIG. 4A, the Bcl-2 expression is shown as the geometric mean fluorescence intensity. In FIG. 4B, the Bcl-2 expression is shown as a fold increase over the corresponding expression in the control HSCs (i.e., no payload and no squeeze processing). The different groups shown are as follows: (1) no payload and no squeeze processing “NC”; (2) squeeze processing without any payload (“empty”); (3) squeeze processing with wild-type Bcl-2 mRNA (“Bcl-2 wt”); and (4) squeeze processing with Bcl-2-G101V variant mRNA (“Bcl-2 G101V”)—i.e., Groups A, B, G, and H, respectively, in Table 6 (see Example 2).

FIGS. 5A and 5B show Bcl-2 expression in squeeze processed human CD34+ HSCs after adoptive transfer into NSG mice. FIG. 5A provides a schematic of the experimental design. As shown, prior to the transfer, the human CD34+ HSCs were squeeze processed with a control mRNA or Bcl-2 variant mRNA (i.e., comprising the G101V amino acid modification). FIG. 5B provides a comparision of Bcl-2 expression (geometric mean fluorescence intensity) by the adoptively transferred cells within the bone marrow at 24 hours post adoptive transfer.

FIGS. 6A and 6B show competitive engraftment of human CD34+ HSCs squeeze processed with a GFP mRNA (“GFP HSCs”) or Bcl-2 variant mRNA (i.e., comprising the G101V amino acid modification) (“Bcl-2 variant HSCs”) after adoptive transfer into NSG mice. FIG. 6A provides a schematic of the experimental design. As shown, the GFP HSCs and the Bcl-2 variant HSCs were administered to the mice at 1:1 ratio, and some of the mice were further dosed with Bcl-2 and MCL-1 inhibitors (i.e., venetoclax and S63845, respectively). FIG. 6B provides a comparison of the frequency of the transferred HSCs (as a percentage of human cells) within the bone marrow of the animals at 24 hours post adoptive transfer.

### DETAILED DESCRIPTION OF DISCLOSURE

The present disclosure is generally directed to methods of producing hematopoietic stem cells (HSCs) that exhibit one or more enhanced properties. Specifically, the methods provided herein comprise passing a cell suspension comprising a population of HSCs through a constriction under one or more parameters. In some aspects, this results in the transient perturbations in the cell membrane of the HSCs, through which a payload can enter and thereby enhance one or more properties of the HSCs (e.g., increased resistance to a mobilization factor, increased resistance to an apoptotic factor, increased resistance to a depleting factor, increased expression of a homing receptor, or a combination thereof). Non-limiting examples of the various aspects are shown in the present disclosure.

### I. General Techniques

Some of the techniques and procedures described or referenced herein are generally well understood and commonly employed using conventional methodology by those skilled in the art, such as, for example, the widely utilized methodologies described in Molecular Cloning: A Laboratory Manual (Sambrook et al., 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons, 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D .G. Newell, eds., J. Wiley and Sons, 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 2011).

### II. Definitions

For purposes of interpreting this specification, the following definitions will apply and whenever appropriate, terms used in the singular will also include the plural and vice versa. In the event that any definition set forth below conflicts with any document incorporated herein by reference, the definition set forth herein shall control. Additional definitions are set forth throughout the detailed description.

As used herein, the singular form term “a,” “an,” and “the” entity refers to one or more of that entity unless indicated otherwise. As such, the terms “a” (or “an” or “the”), “one or more,” and “at least one” can be used interchangeably herein.

It is understood that aspects and aspects of the disclosure described herein include “comprising,” “consisting,” and “consisting essentially of” aspects and aspects. It is also understood that wherever aspects and aspects are described herein with the language “comprising,” otherwise analogous aspects or aspects described in terms of “consisting of” and/or “consisting essentially of” are also provided.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

For all compositions described herein, and all methods using a composition described herein, the compositions can either comprise the listed components or steps, or can “consist essentially of” the listed components or steps. When a composition is described as “consisting essentially of” the listed components, the composition contains the components listed, and can further contain other components which do not substantially affect the methods disclosed, but do not contain any other components which substantially affect the methods disclosed other than those components expressly listed; or, if the composition does contain extra components other than those listed which substantially affect the methods disclosed, the composition does not contain a sufficient concentration or amount of the extra components to substantially affect the methods disclosed. When a method is described as “consisting essentially of” the listed steps, the method contains the steps listed, and can further contain other steps that do not substantially affect the methods disclosed, but the method does not contain any other steps which substantially affect the methods disclosed other than those steps expressly listed. As a non-limiting specific example, when a composition is described as “consisting essentially of” a component, the composition can additionally contain any amount of pharmaceutically acceptable carriers, vehicles, or diluents and other such components which do not substantially affect the methods disclosed.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “apoptosis” refers to the killing of a cell by activation of a programmed cell death pathway. Cells undergoing apoptosis can be generally characterized by morphological and biochemical changes, e.g., DNA fragmentation, chromatin condensation, chromosome migration, margination in cell nuclei, formation of apoptotic bodies, mitochondrial swelling, widening of the mitochondrial cristae, opening of the mitochondrial permeability transition pores, dissipation of the mitochondrial proton gradient, caspase activity, or a combination thereof. The term “apoptotic factor” refers to any agent that is capable of inducing apoptosis in a cell (e.g., HSCs). In some aspects, the apoptotic factor can act directly on the cell to induce apoptosis. In some aspects, the apoptotic factor acts indirectly by inhibiting the activity of an anti-apoptotic factor. As used herein, the term “anti-apoptotic factor” refers to any molecule that is capable of reducing or preventing the apoptosis of a cell. Non-limiting examples of anti-apoptotic factors include Bcl-2, MCL-1, BCL-XL, and combinations thereof. Non-limiting examples of apoptotic factors include an inhibitor of Bcl-2 (“Bcl-2 inhibitor”) (e.g., venetoclax), an inhibitor of MCL-1 (“MCL-1 inhibitor”) (e.g., S63845 and S64315), an inhibitor of BCL-XL (“BCL-XL inhibitor”), or a combination thereof. Such inhibitors are also referred to herein as “inhibitor of an anti-apoptotic factor.”

The term “constriction” as used herein refers to a narrowed passageway. In some aspects, the constriction is a microfluidic channel, such as that contained within a microfluidic device. In some aspects, the constriction is a pore or contained within a pore. Where the constriction is a pore, in some aspects, the pore is contained in a surface. Unless indicated otherwise, the term constriction refers to both microfluidic channels and pores, as well as other suitable constrictions available in the art. Therefore, where applicable, disclosures relating to microfluidic channels can also apply to pores and/or other suitable constrictions available in the art. Similarly, where applicable, disclosures relating to pores can equally apply to microfluidic channels and/or other suitable constrictions available in the art.

As used herein, the terms “deform” and “deformity” (including derivatives thereof) refer to a physical change in a cell. As described herein, as a cell passes through a constriction (such as those of the present disclosure), it experiences various forces due to the constraining physical environment, including but not limited to mechanical deforming forces and/or shear forces that causes perturbations in the cell membrane. As used herein, a “perturbation” within the cell membrane refers to any opening in the cell membrane that is not present under normal steady state conditions (e.g., no deformation force applied to the cells). Perturbation can comprise a hole, tear, cavity, aperture, pore, break, gap, perforation, or combinations thereof.

As used herein, the term “depleting factor” refers to any agent that is capable of inducing the depletion (i.e., removal or deletion) of HSCs from a stem cell niche (e.g., bone marrow) of a subject. As is apparent from the present disclosure, in some aspects, HSCs can be depleted from a stem cell niche by mobilizing the HSCs (e.g., from the bone marrow to the peripheral blood). A non-limiting example of a depleting factor includes CD117 antibody-drug conjugates. In some aspects, HSCs can be depleted by inducing cell death (e.g., by inducing apoptosis of the HSCs). Accordingly, in some aspects, the term “depleting factor” can be used interchangeably with the term “mobilization factor” and/or “apoptotic factor.” Non-limiting examples of additional mobilization factors and apoptotic factors are provided elsewhere in the present disclosure.

The term “filter” as used herein refers to a porous article that allows selective passage through the pores. In some aspects, the term refers to a surface or membrane containing pores.

As used herein, the term “hematopoietic stem cell” or “HSC” refers to a subset of multipotent stem cells that give rise to all the blood or immune cell types, including myeloid (e.g., monocytes and macrophages, neutrophils, basophils, eosinophils, erythrocytes, megakaryocytes/platelets, dendritic cells, mast cells), and lymphoid lineages (e.g., innate lymphoid cells, T-cells, B-cells, NKT-cells, NK-cells), and having multi-lineage hematopoietic differentiation potential and sustained self-renewal activity. Methods of identifying HSCs that can be used with the present disclosure are known in the art. For example, in some aspects, HSCs useful for the present disclosure can be identified based on their phenotypic expression (e.g., with flow cytometry), as they are CD34+, CD90+, CD45RA−, and lack the expression of any lineage markers (CD2, CD3, CD11b, CD11c, CD14, CD16, CD19, CD24, CD56, CD66b, and CD235).

The term “heterogeneous” as used herein refers to something which is mixed or not uniform in structure or composition. For example, when used to describe pores present in a constriction provided herein, such a term can refer to pores having varied sizes, shapes, or distributions within a given surface.

As used herein, the term “homing factor” refers to any agent that is capable of inducing the homing (i.e., migration) of a cell to a particular compartment within a subject. For example, in some aspects, a homing factor provided herein is capable of inducing the HSCs to migrate to the bone marrow when administered to the subject. A non-limiting example of a homing factor comprises a homing receptor. “Homing receptor” refers to a molecule expressed on the surface of a cell (e.g., HSCs) and which exhibits an affinity for a ligand, such that the cell expressing the homing receptor preferentially migrates towards the ligand. As further described elsewhere in the present disclosure, in some aspects, a homing receptor is a chemokine receptor, such as CXCR4 and CXCR2.

The term “homogeneous” as used herein refers to something which is consistent or uniform in structure or composition throughout. For example, when used to describe pores present in a constriction provided herein, such a term can refer to pores having consistent sizes, shapes, or distribution within a given surface.

The term “membrane” as used herein refers to a selective barrier or sheet containing pores. The term includes, but is not limited to, a pliable sheet-like structure that acts as a boundary or lining. In some aspects, the term refers to a surface or filter containing pores. This term is distinct from the term “cell membrane,” which refers to a semipermeable membrane surrounding the cytoplasm of cells.

As used herein, the term “mobilization” (or grammatical derivatives thereof) refers to the recruitment of HSCs from a first location (e.g., stem cell niche, e.g., bone marrow) to a second location (e.g., tissue, peripheral blood, or organ). In some aspects, the first location is the bone marrow and the second location is the peripheral blood. As used herein, the term “mobilization factor” comprises any agent that is capable of inducing the mobilization of HSCs (e.g., from the bone marrow to the peripheral blood). Non-limiting examples of mobilization factors include: plerixafor (e.g., MOZOBIL®), AMD3465 (CXCR4 antagonist), Groβ (CXCR2 agonist, induction of MMP-9 secretion), granulocyte colony-stimulating factor (G-CSF), anti-CD117 (c-kit) antibody, chemotherapy (e.g., cyclophosphamide, etoposide (e.g., TOPOSAR® and ETOPOPHOS®), POL6326 (CXCR4 antagonist), TG-0054 (CXCR4 antagonist), BKT140 (anti-SDF-1), bortezomib (proteasome inhibitor, downregulation of VLA4/VCAM-1 axis) (e.g., VELCADE®), PTH (PTH receptor agonist, expansion of BM HSC), CDX-301 (FLT3 agonist), LY2510924 (CXCR4 antagonist), natalizumab (VLA-4 antagonist) (e.g., TYSABRI®), meloxicam (Non-steroidal anti-inflammatory drug) (e.g., VIVLODEX®, MOBIC®, COMFORT®), eltrombopag (TPO receptor agonist) (e.g., PROMACTA®), ALX-0651 (Anti-CXCR4 Nanobody), and combinations thereof. See, e.g., Domingues et al., Int J Hematol 105:141-152 (2017); Bakanay et al., Bone Marrow Transplantation 47:1154-1163 (2012), both of which is herein incorporated by reference in its entirety. or a combination thereof.

The term “polynucleotide” or “nucleic acid” as used herein refers to a polymeric form of nucleotides of any length, either ribonucleotides or deoxyribonucleotides. Thus, this term includes, but is not limited to, single-, double-or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as can typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues can contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications can be deliberate, as through site-directed mutagenesis, or can be accidental, such as through mutations of hosts which produce the proteins or errors due to PCR amplification.

The term “pore” as used herein refers to an opening, including without limitation, a hole, tear, cavity, aperture, break, gap, or perforation within a material. In some aspects, (where indicated) the term refers to a pore within a surface of a microfluidic device, such as those described in the present disclosure. In some aspects, (where indicated) a pore can refer to a pore in a cell wall and/or cell membrane.

As used herein, the term “reference” HSC refers to a HSC that has not been modified using the squeeze processing methods provided herein. For example, in some aspects, a reference HSC refers to a corresponding HSC that has been exposed to the payload but using alternative delivery methods known in the art (e.g., electroporation or lipofection). In some aspects, a reference HSC refers to a corresponding HSC which has undergone squeeze processing but without any of the payloads described herein. In some aspects, a reference HSC refers to a corresponding HSC which has not been modified (i.e., no squeeze processing and no delivery of any payload).

As used herein, the term “survival factor” refers to any agent that is capable of promoting the survival of a cell (e.g., HSC). Such agents are also referred to herein as “anti-apoptotic factor.” For example, in some aspects, a survival factor can reduce or prevent a cell from undergoing apoptosis. Non-limiting examples of survival factors include Bcl-2, Bcl-xL, MCL-1, Ced-9, bfl-1, or a combination thereof. As is apparent from the present disclosure, modifying a HSC to exhibit increased expression of a survival factor through the squeeze processing methods provided herein can result in increased survival of the HSC compared to a reference HSC.

### III. Methods of the Disclosure

In some aspects, the present disclosure relates to methods of producing HSCs with enhanced properties by delivering a payload into the HSCs, wherein the payload is capable of enhancing one or more properties of the HSCs. As further described herein, such methods comprise passing a cell suspension, which comprises a population of the HSCs, through a constriction under one or more parameters. As the cells pass through the constriction, they become transiently deformed, such that cell membrane of the HSC is perturbed. The perturbations within the cell membrane can allow the payload to enter or be loaded into the cell (e.g., through diffusion). The specific process by which the cells pass through a constriction and become transiently deformed is referred to herein as “squeeze processing” or “squeezing.”

As further described herein, the squeeze processing methods of the present disclosure have certain distinct properties that are not shared by other delivery methods known in the art. For example, in addition to the improved ability to deliver various types of payloads into a cell, the squeeze processing methods described herein exert minimal lasting effects on the cells. Compared to traditional delivery methods such as electroporation, the squeeze processing methods of the present disclosure preserve both the structural and functional integrity of the squeezed cells. Contrary to the delivery methods provided herein, electroporation can induce broad and lasting alterations in gene expression, which can lead to non-specific activation of cells (e.g., human T cells) and delayed proliferation upon antigen stimulation. With the present methods, any alterations to the cells (e.g., perturbations in the cell membrane) is transient and quickly repaired once the cells are removed from the constriction.

As demonstrated herein, using such improved delivery methods, different aspects of a HSC can be modified, such that the HSCs described herein are structurally and functionally distinct from corresponding cells that naturally exist in nature. For example, in some aspects, by using a payload that plays an important role in the engraftment of HSCs within the bone marrow of a subject (e.g., promotes homing to the bone marrow and/or increases survival) (e.g., CXCR4 and/or Bcl-2), the squeeze processing methods provided here can be used to express (e.g., overexpress) such payloads in the HSCs and thereby, allow for better engraftment of the HSCs when administered to the subject.

Additionally, in some aspects, by using a payload that is capable of increasing resistance to a mobilization factor, the squeeze processing methods provided herein can enhance the ability of HSCs to resist the effects of a mobilization factor. Similarly, in some aspects, by using a payload that is capable of increasing resistance of the HSC to an apoptotic factor, the squeeze processing methods of the present disclosure can enhance the ability of the HSCs to resist the effects of an apoptotic factor. And, by using a payload that is capable of increasing the resistance to a HSC depleting factor, the squeeze processing methods provided herein can be used to produce HSCs with increased resistance to such depleting factors. As further described elsewhere in the present disclosure, such enhanced properties can be particularly useful as they can be engrafted within the bone marrow of a subject without the need for harsh and toxic myeloablative preconditioning.

While the present disclosure generally discloses the use of payloads that are capable of increasing the specific properties described above (i.e., resistance of the HSCs to a mobilization factor, resistance of the HSCs to an apoptotic factor, resistance of the HSCs to a depleting factor, and/or increased expression of a homing factor) it will be apparent to those skilled in the art that disclosures related to such payloads can equally apply to other types of payloads, e.g., those that enhance one or more other properties of the HSCs.

In some aspects, provided herein is a method of producing a modified HSC, wherein the method comprises passing a cell suspension through a constriction under one or more parameters, wherein the cell suspension comprises a population of HSCs, wherein passing the cell suspension through the constriction under the one or more parameters allows a payload to enter the HSC (e.g., through transient perturbations created in the cell membrane of the HSC as the cell suspension passes through the constriction), and wherein the payload is capable of modifying the HSC, such that the HSC exhibits: (i) increased resistance to a mobilization factor, (ii) increased resistance to an apoptotic factor, (iii) increased resistance to a depleting factor, (iv) increased expression of a homing factor, or (v) any combination thereof. Non-limiting examples of such payloads are provided elsewhere in the present disclosure.

In some aspects, compared to a reference HSC, the modified HSCs produced using the methods provided herein are more resistant to a mobilization factor by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold. In some aspects, compared to a reference HSC, the HSCs produced using the methods provided herein are more resistant to an apoptotic factor by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold. In some aspects, compared to a reference HSC, the HSCs produced using the methods provided herein are more resistant to a depleting factor by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold. In some aspects, compared to a reference HSC, the HSCs produced using the methods provided herein have increased expression of a homing factor. In some aspects, the expression of the homing factor is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold.

Accordingly, in some aspects, the methods provided herein can be used to increase the homing of a HSC when administered to a subject in need thereof (e.g., as a result of the increased resistance to a mobilization factor, increased resistance to a depletion factor, and/or increased expression of a homing factor). In some aspects, such a method comprises passing a cell suspension, which comprises the HSC, through a constriction under one or more parameters, such that a payload enters the HSC (e.g., through transient perturbations created in the cell membrane of the HSC as the cell suspension passes through the constriction), wherein the payload is capable of modifying the HSC, such that the HSC exhibits increased homing to the bone marrow when administered to the subject.

In some aspects, the methods provided herein can also be used to increase the survival of a HSC when administered to a subject in need thereof (e.g., as a result of the increased resistance to an apoptotic factor). In some aspects, such a method comprising passing a cell suspension, which comprises the HSC, through a constriction under one or more parameters, such that a payload enters the HSC (e.g., through transient perturbations created in the cell membrane of the HSC as the cell suspension passes through the constriction), wherein the payload is capable of modifying the HSC, such that the HSC exhibits increased survival when administered to the subject.

As will be apparent to those skilled in the arts and further described elsewhere in the present disclosure (see, e.g., section titled “Therapeutic Uses”), the above methods have various important clinical implications. For example, in some aspects, by enhancing one or more properties of HSCs described herein, the engraftment of HSCs when administered to a subject can be improved. Accordingly, in some aspects, the present disclosure is directed to a method of promoting the engraftment of a HSC within the bone marrow of a subject in need thereof, comprising passing a cell suspension, which comprises the HSC, through a constriction under one or more parameters, such that a payload enters the HSC (e.g., through transient perturbations created in the cell membrane of the HSC as the cell suspension passes through the constriction), wherein the payload is capable of modifying the HSC, such that the HSC is engrafted within the bone marrow of the subject when administered to the subject.

Compared with reference HSCs, the methods provided herein can result in increased engraftment of the administered HSCs. In some aspects, engraftment of the administered HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to engraftment observed after administration of the reference HSCs.

In some aspects, the above methods further comprise contacting the HSCs with a payload (e.g., described herein) prior to passing the cell suspension, which comprises the HSCs, through the constriction. As is apparent from the present disclosure, in some aspects, contacting the HSCs with the payload prior to the squeezing can help delivery efficiency, as the payload would be able to enter the cell as soon as the perturbations in the cell membrane are created through the squeeze processing. In some aspects, prior to passing the cell suspension through the constriction, the methods provided herein comprise contacting the HSCs with the payload to produce the cell suspension. In some aspects, the methods provided herein comprise contacting the HSCs with the payload as the cell suspension passes through the constriction. In some aspects, the HSCs are first contacted with the payload during the passing of the cell suspension through the constriction. In some aspects, the HSCs are in contact with the payload both prior to the passing step (i.e., passing of the cell suspension through the constriction) and during the passing step. In some aspects, the methods provided herein comprise contacting the HSCs with the payload after the passing of the cell suspension through the constriction. In some aspects, the cells are first contacted with the payload after the passing of the cell suspension through the constriction. In some aspects, the HSCs are in contact with the payload prior to, during, and/or, after the passing step. As further described elsewhere in the present disclosure, when the HSCs are contacted with the payload after the passing step, the contacting occurs soon after the HSCs have passed through the constriction, such that there are still perturbations within the cell membrane.

As used herein, the “contacting” that can occur between a HSC and a payload (e.g., described herein) includes that a cell can be in contact with the payload as long as the payload is capable of entering the cell once there are perturbations within the cell membrane of the cell. To help illustrate, in some aspects, a cell and a payload are in contact if they are both present within the same cell suspension.

### III.A. Cell Suspensions

In some aspects, a cell suspension described herein comprises any suitable HSCs known in the art that can be modified (e.g., by introducing a payload) using the squeeze processing methods described herein. While the present application generally describes the use of HSCs, it will be apparent to those skilled in the arts that the disclosures provided herein can be applicable for other types of stem cells that can be useful in treating a disease or disorder described herein (e.g., blood disorder). As used herein, the term “stem cells” refer to cells having not only self-replication ability but also the ability to differentiate into other types of cells. In some aspects, stem cells useful for the present disclosure comprise induced pluripotent stem cells (iPSCs), embryonic stem cells (ESCs), tissue-specific stem cells (e.g., liver stem cells, cardiac stem cells, or neural stem cells), mesenchymal stem cells, hematopoietic stem cells (HSCs), or combinations thereof. In some aspects, the stem cells are HSCs.

As demonstrated herein, the delivery of a payload into a cell can be regulated through one or more parameters of the process in which a cell suspension is passed through a constriction. In some aspects, the specific characteristics of the cell suspension can impact the delivery of a payload into a cell. Such characteristics include, but are not limited to, osmolarity, salt concentration, serum content, cell concentration, pH, temperature or combinations thereof. Additional parameters relevant for the present disclosure are provided elsewhere in the present disclosure.

In some aspects, the cell suspension comprises a homogeneous population of cells (e.g., purified population of HSCs). In some aspects, the cell suspension comprises a heterogeneous population of cells (e.g., whole blood or a mixture of cells comprising HSCs, e.g., in a physiological saline solution or physiological medium other than blood). Where the cell suspension comprises a heterogeneous population of cells, in some aspects, the HSCs can constitute about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 60%, about 70%, about 80%, or about 90% of the total population of cells. In some aspects, a heterogeneous population of cells has undergone an enrichment step, such that the HSCs make up a greater percentage of the total cell population.

In some aspects, the cell suspension comprises an aqueous solution. In some aspects, the aqueous solution comprises a cell culture medium, PBS, salts, sugars, growth factors, animal derived products, bulking materials, surfactants, lubricants, vitamins, polypeptides, an agent that impacts actin polymerization, or combinations thereof. In some aspects, the cell culture medium comprises DMEM, OptiMEM, EVIDM, RPMI, XVivo10, or combinations thereof. Additionally, solution buffer can include one or more lubricants (pluronics or other surfactants) that can be designed to reduce or eliminate clogging of the surface and improve cell viability. Exemplary surfactants include, without limitation, poloxamer, polysorbates, sugars such as mannitol, animal derived serum, and albumin protein.

When the suspension includes certain types of cells, in some aspects, the cells can be treated with a solution that aids in the delivery of the payload to the interior of the cell. In some aspects, the solution comprises an agent that impacts actin polymerization. In some aspects, the agent that impacts actin polymerization comprises Latrunculin A, Cytochalasin, Colchicine, or combinations thereof. For example, in some aspects, the cells can be incubated in a depolymerization solution, such as Lantrunculin A, for about 1 hour prior to passing the cells through a constriction to depolymerize the actin cytoskeleton. In some aspects, the cells can be incubated in Colchicine (Sigma) for about 2 hours prior to passing the cells through a constriction to depolymerize the microtubule network.

In some aspects, a characteristic of a cell suspension that can affect the delivery of a payload (into a cell is the viscosity of the cell suspension. As used herein, the term “viscosity” refers to the internal resistance to flow exhibited by a fluid. In some aspects, the viscosity of the cell suspension is between about 8.9×10⁻⁴ Pa·s to about 4.0×10⁻³ Pa·s, between about 8.9×10⁻⁴ Pa·s to about 3.0×10⁻³ Pa·s, between about 8.9×10⁻⁴ Pa·s to about 2.0×10⁻³ Pa·s, or between about 8.9×10⁻⁴ Pa·s to about 1.0×10⁻³ Pa·s. In some aspects, the viscosity is between about 0.89 cP to about 4.0 cP, between about 0.89 cP to about 3.0 cP, between about 0.89 cP to about 2.0 cP, or between about 0.89 cP to about 1.0 cP. In some aspects, a shear thinning effect is observed, in which the viscosity of the cell suspension decreases under conditions of shear strain. Viscosity can be measured by any suitable method known in the art, including without limitation, viscometers, such as a glass capillary viscometer or rheometers. A viscometer measures viscosity under one flow condition, while a rheometer is used to measure viscosities which vary with flow conditions. In some aspects, the viscosity is measured for a shear thinning solution such as blood. In some aspects, the viscosity is measured between about 0° C. and about 45° C. For example, the viscosity of the cell suspension can be measured at room temperature (e.g., about 20° C.), physiological temperature (e.g., about 37° C.), higher than physiological temperature (e.g., greater than about 37° C. to about 45° C. or more), reduced temperature (e.g., about 0° C. to about 4° C.), or temperatures between these exemplary temperatures.

### III.B. Payloads

As described herein, in some aspects, a cell suspension additionally comprises one or more payloads, such as those that can improve one or more properties of the HSCs such that the HSCs can better engraft when administered to a subject (e.g., increase the resistance of HSCs to a mobilization factor, increase the resistance of the HSCs to an apoptotic factor, increase the resistance of the HSCs to a depletion factor, increase the expression of a homing factor in the HSCs, or a combination thereof). Non-limiting examples of payloads useful for the present disclosure comprises a homing factor (e.g., homing receptor), a cytokine, a growth factor, a cell adhesion molecule, a proliferative agent, a survival factor, or a combination thereof. In some aspects, a payload that can be used with the present disclosure comprises a regulator of such payloads. The term “regulator” when used to describe a payload useful for the present disclosure refers to any agent that can modulate (e.g., increase and/or decrease) the expression and/or activity of the other payloads described herein. For instance, in some aspects, a payload useful for the present disclosure comprises a regulator for a homing factor. In some aspects, a payload that can be used with the present disclosure comprises a regulator for a cytokine. In some aspects, a payload comprises a regulator of a growth factor. In some aspects, a payload comprises a regulator of a cell adhesion molecule. In some aspects, a payload comprises a regulator for a proliferative agent. In some aspects, a payload comprises a regulator for a survival factor. Additional aspects of such payloads are described further below.

As is apparent from the present disclosure, in some aspects, the payloads useful for the present disclosure are transiently expressed in the modified HSCs, such that any modifications to one or more properties of the HSCs mediated by the payload are not permanent (i.e., transiently expressed). For instance, in some aspects, the payload is expressed in the HSCs for less than about one day, less than about two days, less than about three days, less than about four days, less than about five days, less than about six days, less than about seven days, less than about eight days, less than about nine days, or less than about 10 days. In some aspects, the payload is expressed in the HSCs for about one day, about two days, about three days, about four days, about five days, about six days, about seven days, about eight days, about nine days, or about 10 days.

As described and demonstrated herein, in some aspects, a payload that can be used to produce the modified HSCs described herein comprises a homing factor. For example, in some aspects, the payload comprises a homing receptor, such as that involved in the homing and repopulation of HSCs within the bone marrow. Non-limiting examples of homing receptors useful for the present disclosure include CXCR4, CXCR2, or both. In some aspects, the homing receptor is CXCR4. CXCR4 interacts with CXCL12, which is primarily secreted by mesenchymal stromal cells present within the bone marrow niche. This interaction has been described to be important in both the homing and retention of HSCs within the bone marrow niche. See, e.g., Karpova et al., Stem Cell 33:2391-2399 (2015); and Sugiyama et al., Immunity 25:977-988 (2006).

Accordingly, in some aspects, using the squeeze processing methods provided herein, the expression of CXCR4 on the HSCs can be increased compared to a reference HSC. In some aspects, the expression of CXCR4 on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC. In some aspects, the increased CXCR4 expression on the HSCs can help increase the homing and/or retention of the HSCs within the bone marrow niche.

In some aspects, the CXCR4 can be modified to help further improve one or more properties of the HSCs produced using the methods provided herein. For example, as will be apparent to those skilled in the arts, to promote the engraftment of any adoptively transferred HSCs (e.g., as part of a HSC transplantation therapy), it is necessary to deplete/reduce the endogenous stem cell compartment to make room for the adoptively transferred HSCs. Plerixafor is a FDA-approved small molecule (i.e., mobilization factor) that inhibits the interaction between CXCR4 and CXCL12, and thereby, promote the mobilization of HSCs from the bone marrow to the peripheral blood. Accordingly, in some aspects, a payload useful for the present disclosure comprises a CXCR4 variant which comprises one or more mutations that are capable of conferring resistance to plerixafor or any other similar mobilization factors. Non-limiting example of such a CXCR4 variant comprises the mutation A175F as compared to the corresponding wild-type amino acid sequence set forth in SEQ ID NO: 1 (see Tables 1 and 2).

TABLE 1
CXCR4 Variant Amino Acid Sequences

| | |
|---|---|
| Wild-type human | MEGISIYTSDNYTEEMGSGDYDSMKEPCFREENANFNKIFLPTIYSI |
| CXCR4 (SEQ ID NO: | IFLTGIVGNGLVILVMGYQKKLRSMTDKYRLHLSVADLLFVITLPFW |
| 1) | AVDAVANWYFGNFLCKAVHVIYTVNLYSSVLILAFISLDRYLAIVHA |
| | TNSQRPRKLLAEKVVYVGVWIPALLLTIPDFIFANVSEADDRYICDR |
| | FYPNDLWVVVFQFQHIMVGLILPGIVILSCYCIIISKLSHSKGHQKR |
| | KALKTTVILILAFFACWLPYYIGISIDSFILLEIIKQGCEFENTVHK |
| | WISITEALAFFHCCLNPILYAFLGAKFKTSAQHALTSVSRGSSLKIL |
| | SKGKRGGHSSVSTESESSSFHSS |
| Human CXCR4 | MEGISIYTSDNYTEEMGSGDYDSMKEPCFREENANFNKIFLPTIYSI |
| A175F Variant (SEQ | IFLTGIVGNGLVILVMGYQKKLRSMTDKYRLHLSVADLLFVITLPFW |
| ID NO: 2) | AVDAVANWYFGNFLCKAVHVIYTVNLYSSVLILAFISLDRYLAIVHA |
| | TNSQRPRKLLAEKVVYVGVWIPALLLTIPDFIFFNVSEADDRYICDR |
| | FYPNDLWVVVFQFQHIMVGLILPGIVILSCYCIIISKLSHSKGHQKR |
| | KALKTTVILILAFFACWLPYYIGISIDSFILLEIIKQGCEFENTVHK |
| | WISITEALAFFHCCLNPILYAFLGAKFKTSAQHALTSVSRGSSLKIL |
| | SKGKRGGHSSVSTESESSSFHSS |
| Human CXCR4 | MEGISIYTSDNYTEEMGSGDYDSMKEPCFREENANFNKIFLPTIYSI |
| R334X Variant (SEQ | IFLTGIVGNGLVILVMGYQKKLRSMTDKYRLHLSVADLLFVITLPFW |
| ID NO: 3) | AVDAVANWYFGNFLCKAVHVIYTVNLYSSVLILAFISLDRYLAIVHA |
| | TNSQRPRKLLAEKVVYVGVWIPALLLTIPDFIFANVSEADDRYICDR |
| | FYPNDLWVVVFQFQHIMVGLILPGIVILSCYCIIISKLSHSKGHQKR |
| | KALKTTVILILAFFACWLPYYIGISIDSFILLEIIKQGCEFENTVHK |
| | WISITEALAFFHCCLNPILYAFLGAKFKTSAQHALTSVSRGSSLKIL |
| | SKGK |
| Human CXCR4 | MEGISIYTSDNYTEEMGSGDYDSMKEPCFREENANFNKIFLPTIYSI |
| A175F + R334X | IFLTGIVGNGLVILVMGYQKKLRSMTDKYRLHLSVADLLFVITLPFW |
| Variant (SEQ ID NO: | AVDAVANWYFGNFLCKAVHVIYTVNLYSSVLILAFISLDRYLAIVHA |
| 4) | TNSQRPRKLLAEKVVYVGVWIPALLLTIPDFIFFNVSEADDRYICDR |
| | FYPNDLWVVVFQFQHIMVGLILPGIVILSCYCIIISKLSHSKGHQKR |
| | KALKTTVILILAFFACWLPYYIGISIDSFILLEIIKQGCEFENTVHK |
| | WISITEALAFFHCCLNPILYAFLGAKFKTSAQHALTSVSRGSSLKIL |
| | SKGK |

TABLE 2
CXCR4 Variant Nucleotide Sequences

| | |
|---|---|
| Wild-type human | ATGGAAGGCATCAGCATCTACACCAGCGACAACTACACAGAGGAAAT |
| CXCR4 (SEQ ID NO: | GGGCAGCGGCGACTACGACAGCATGAAAGAGCCTTGTTTCCGGGAAG |
| 5) | AGAACGCCAATTTCAACAAGATTTTCCTGCCAACAATCTATAGCATC |
| | ATCTTTCTGACCGGCATCGTGGGAAATGGCCTGGTGATCCTGGTCAT |
| | GGGCTACCAGAAGAAGCTGAGATCTATGACAGATAAGTACCGGCTGC |
| | ACCTGAGCGTGGCTGATCTGCTGTTCGTGATCACCCTGCCCTTCTGG |
| | GCCGTGGACGCCGTGGCCAACTGGTATTTCGGCAACTTCCTGTGCAA |
| | GGCCGTGCACGTGATCTACACCGTGAACCTGTACAGCTCCGTGCTGA |
| | TCCTGGCTTTTATCAGCCTGGATAGATACCTGGCCATCGTGCACGCC |
| | ACCAACTCACAGAGACCTAGAAAGCTGCTGGCCGAGAAGGTGGTCTA |
| | CGTGGGAGTGTGGATCCCTGCCCTGCTGCTCACCATCCCCGACTTCA |
| | TCTTCGCCAACGTGTCCGAGGCCGACGACCGGTACATCTGCGACAGA |
| | TTCTACCCCAACGACCTGTGGGTCGTGGTGTTCCAGTTCCAACACAT |
| | CATGGTGGGCCTGATTCTGCCTGGCATCGTGATCCTGAGCTGCTACT |
| | GCATCATCATCAGCAAGCTGTCCCACAGCAAAGGCCACCAGAAAAGA |
| | AAGGCCCTGAAAACCACCGTGATCCTGATCCTCGCTTTTTTCGCGTG |
| | CTGGCTGCCTTACTACATCGGCATTAGCATCGATAGCTTCATCCTGC |
| | TGGAAATCATCAAGCAGGGTTGTGAATTCGAGAACACCGTGCATAAG |
| | TGGATCTCTATTACAGAGGCCCTGGCTTTCTTCCACTGCTGTCTGAA |
| | TCCTATCCTGTACGCCTTTCTGGGAGCAAAGTTCAAGACCAGCGCCC |
| | AGCACGCCCTGACCTCTGTTTCTCGGGGAAGCAGCCTGAAGATCCTC |
| | TCCAAGGGCAAGAGAGGCGGCCACAGCAGCGTGTCGACAGAGAGCGA |
| | GAGCAGCTCTTTTCATTCTTCT |
| Human CXCR4 | ATGGAAGGCATCAGCATCTATACATCCGACAACTACACAGAGGAAAT |
| A175F Variant (SEQ | GGGATCTGGCGACTACGACAGCATGAAAGAGCCTTGTTTCCGGGAAG |
| ID NO: 6) | AGAATGCCAACTTCAACAAGATCTTCTTGCCTACAATCTACTCCATC |
| | ATCTTTCTGACCGGCATCGTGGGAAACGGCCTGGTGATCCTCGTGAT |
| | GGGCTACCAGAAAAAGCTTAGAAGCATGACCGATAAGTACAGACTGC |
| | ACCTGAGCGTCGCCGATCTGCTGTTCGTGATCACACTGCCATTCTGG |
| | GCCGTGGACGCCGTGGCCAATTGGTACTTCGGCAACTTCCTGTGCAA |
| | GGCCGTGCATGTGATCTATACAGTGAACCTGTACAGCAGCGTCCTGA |
| | TCCTGGCTTTTATCTCTCTGGATCGGTACCTGGCTATCGTGCACGCC |
| | ACCAACAGCCAGAGACCTAGAAAGCTGCTGGCTGAGAAGGTGGTGTA |
| | CGTGGGAGTGTGGATCCCCGCCCTGCTGCTGACCATCCCTGATTTCA |
| | TCTTCTTCAATGTGTCTGAGGCCGACGACAGATACATCTGCGACAGG |
| | TTCTACCCCAACGACCTGTGGGTGGTCGTGTTCCAATTTCAGCACAT |
| | CATGGTGGGCCTGATCCTGCCTGGCATCGTTATTCTGAGCTGTTACT |
| | GCATCATTATCTCCAAGCTGTCTCACAGCAAGGGACACCAGAAGCGG |
| | AAGGCCCTCAAAACCACCGTGATCCTGATCCTGGCCTTCTTCGCCTG |
| | CTGGCTGCCTTACTACATCGGCATCAGCATCGACAGCTTTATCCTGC |
| | TGGAAATCATTAAGCAGGGCTGTGAATTCGAGAACACCGTGCACAAG |
| | TGGATCAGCATCACCGAGGCTCTGGCTTTCTTCCACTGCTGCCTGAA |
| | CCCCATCCTGTACGCCTTTCTGGGCGCCAAGTTCAAGACCAGCGCCC |
| | AGCACGCCCTGACAAGCGTGTCCAGAGGCTCTAGCCTGAAAATCCTG |
| | TCCAAGGGCAAGAGAGGCGGCCACAGCAGCGTGAGCACCGAGAGCGA |
| | GAGCTCTAGCTTCCACTCCAGCTGA |
| Human CXCR4 | ATGGAAGGCATCAGCATCTACACCAGCGACAACTACACAGAGGAAAT |
| R334X Variant (SEQ | GGGCAGCGGCGACTACGACAGCATGAAAGAGCCTTGTTTCCGGGAAG |
| ID NO: 7) | AGAACGCCAATTTCAACAAGATTTTCCTGCCAACAATCTATAGCATC |
| | ATCTTTCTGACCGGCATCGTGGGAAATGGCCTGGTGATCCTGGTCAT |
| | GGGCTACCAGAAGAAGCTGAGATCTATGACAGATAAGTACCGGCTGC |
| | ACCTGAGCGTGGCTGATCTGCTGTTCGTGATCACCCTGCCCTTCTGG |
| | GCCGTGGACGCCGTGGCCAACTGGTATTTCGGCAACTTCCTGTGCAA |
| | GGCCGTGCACGTGATCTACACCGTGAACCTGTACAGCTCCGTGCTGA |
| | TCCTGGCTTTTATCAGCCTGGATAGATACCTGGCCATCGTGCACGCC |
| | ACCAACTCACAGAGACCTAGAAAGCTGCTGGCCGAGAAGGTGGTCTA |
| | CGTGGGAGTGTGGATCCCTGCCCTGCTGCTCACCATCCCCGACTTCA |
| | TCTTCGCCAACGTGTCCGAGGCCGACGACCGGTACATCTGCGACAGA |
| | TTCTACCCCAACGACCTGTGGGTCGTGGTGTTCCAGTTCCAACACAT |
| | CATGGTGGGCCTGATTCTGCCTGGCATCGTGATCCTGAGCTGCTACT |
| | GCATCATCATCAGCAAGCTGTCCCACAGCAAAGGCCACCAGAAAAGA |
| | AAGGCCCTGAAAACCACCGTGATCCTGATCCTCGCTTTTTTCGCGTG |
| | CTGGCTGCCTTACTACATCGGCATTAGCATCGATAGCTTCATCCTGC |
| | TGGAAATCATCAAGCAGGGTTGTGAATTCGAGAACACCGTGCATAAG |
| | TGGATCTCTATTACAGAGGCCCTGGCTTTCTTCCACTGCTGTCTGAA |
| | TCCTATCCTGTACGCCTTTCTGGGAGCAAAGTTCAAGACCAGCGCCC |
| | AGCACGCCCTGACCTCTGTTTCTCGGGGAAGCAGCCTGAAGATCCTC |
| | TCCAAGGGCAATGA |
| Human CXCR4 | ATGGAAGGCATCTCCATCTACACCTCTGATAACTACACAGAGGAGAT |
| A175F + R334X | GGGCAGCGGCGACTACGACAGCATGAAGGAACCTTGTTTCCGGGAAG |
| Variant (SEQ ID NO: | AGAACGCCAATTTCAACAAGATCTTCCTGCCTACAATCTACAGCATC |
| 8) | ATCTTCCTCACAGGCATCGTGGGCAACGGCCTGGTGATCCTGGTGAT |
| | GGGATATCAGAAAAAGCTTAGATCCATGACCGATAAGTACAGACTGC |
| | ACCTGAGCGTGGCTGATCTGCTGTTCGTGATCACCCTGCCATTCTGG |
| | GCCGTGGACGCTGTGGCCAACTGGTACTTCGGCAATTTCCTGTGCAA |
| | GGCCGTGCACGTGATCTACACCGTAAACCTGTACAGCAGCGTGCTGA |
| | TCCTGGCTTTTATCTCTCTGGATAGATACCTGGCTATCGTGCATGCC |
| | ACCAACAGCCAGAGGCCTAGAAAGCTGCTGGCCGAGAAGGTGGTGTA |
| | CGTGGGTGTTTGGATCCCTGCCTTGCTGCTGACAATCCCCGACTTCA |
| | TCTTCTTCAATGTGTCTGAGGCCGACGACAGATATATCTGCGACCGG |
| | TTCTACCCCAACGACCTGTGGGTCGTGGTGTTCCAATTTCAGCACAT |
| | CATGGTCGGACTGATCCTGCCTGGCATCGTGATCCTGAGCTGCTACT |
| | GCATCATTATTTCTAAACTGAGCCACAGCAAGGGCCACCAGAAGCGG |
| | AAAGCCCTGAAGACCACCGTGATCCTGATCCTGGCTTTTTTCGCCTG |
| | TTGGCTGCCCTACTACATCGGCATCAGCATCGACTCCTTCATCCTGC |
| | TGGAAATCATTAAGCAGGGCTGTGAATTCGAGAACACCGTGCACAAG |
| | TGGATCAGCATTACCGAGGCCCTGGCCTTCTTTCACTGCTGCCTGAA |
| | CCCTATCCTGTACGCCTTTCTGGGCGCCAAGTTCAAGACCAGCGCCC |
| | AGCACGCCCTGACAAGCGTGTCCAGAGGATCTTCTCTGAAGATCCTG |
| | AGCAAGGGCAAATGA |

It will be apparent to those skilled in the arts by modifying the HSCs to express such a CXCR4 variant, in some aspects, plerixafor can be used to selectively mobilize the non-modified HSCs (e.g., endogenous HSCs) from the bone marrow and thus, allowing for improved engraftment of the adoptively transferred modified HSCs.

In some aspects, the CXCR4 variant can comprise one or more mutations that enhance the function of CXCR4 (e.g., enhancing the interaction between CXCR4 and CXCL12). Non-limiting example of such a CXCR4 variant comprises the mutation R334X as compared to the corresponding wild-type amino acid sequence set forth in SEQ ID NO: 1 (see Table 1). In some aspects, the CXCR4 variant comprises both the A175F and R334X mutations as compared to the corresponding wild-type amino acid sequence set forth in SEQ ID NO: 1. Non-limiting examples of additional mutations that a CXCR4 variant described herein include H113A, D17IN, D262N, I284A, H281A, Q200W, Q200A, or a combination thereof.

In some aspects, the homing receptor is CXCR2 or a variant thereof. In some aspects, using the squeeze processing methods provided herein, the expression of CXCR2 on the HSCs can be increased. In some aspects, compared to a reference HSC, the expression of CXCR2 can be increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold.

In some aspects, a payload that is useful for the present disclosure comprises a survival factor, such as those that can increase the survival of a HSC when expressed (or overexpressed) in the HSC. Non-limiting examples of survival factors include Bcl-2, Bcl-xL, MCL-1, Ced-9, bfl-1, or a combination thereof.

In some aspects, the payload comprises a Bcl-2. Qing et al., Blood 123:1002 (2014). Accordingly, in some aspects, using the squeeze processing methods provided herein, the expression of Bcl-2 on the HSCs can be increased compared to a reference HSC. In some aspects, the expression of Bcl-2 on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC.

In some aspects, the Bcl-2 can be modified to help further improve one or more properties of the HSCs produced using the methods provided herein. Venetoclax is a FDA-approved BH3 mimetic which specifically targets and inhibits Bcl-2. Accordingly, in some aspects, a payload useful for the present disclosure comprises a Bcl-2 variant which comprises one or more mutations that are capable of conferring resistance to venetoclax or any other similar apoptotic factors. Non-limiting example of such a Bcl-2 variant comprises the mutation G101V, D103Y, or both, as compared to the corresponding wild-type amino acid sequence set forth in SEQ ID NO: 9 (see Tables 3 and 4). It will be apparent to those skilled in the arts by modifying the HSCs to express such a Bcl-2 variant, in some aspects, venetoclax can be used to selectively deplete the non-modified HSCs (e.g., endogenous HSCs) from the bone marrow and thus, allowing for improved engraftment of the adoptively transferred modified HSCs.

TABLE 3
Bcl-2 Variant Amino Acid Sequences

| | |
|---|---|
| Wild-type human | MAHAGRTGYDNREIVMKYIHYKLSQRGYEWDAGDVGAAPPGAAPAPG |
| Bcl-2 (SEQ ID NO: 9) | IFSSQPGHTPHPAASRDPVARTSPLQTPAAPGAAAGPALSPVPPVVH |
| | LTLRQAGDDFSRRYRRDFAEMSSQLHLTPFTARGRFATVVEELFRDG |
| | VNWGRIVAFFEFGGVMCVESVNREMSPLVDNIALWMTEYLNRHLHTW |
| | IQDNGGWDAFVELYGPSMRPLFDFSWLSLKTLLSLALVGACITLGAY |
| | LGHK |
| Human Bcl-2 G101V | MAHAGRTGYDNREIVMKYIHYKLSQRGYEWDAGDVGAAPPGAAPAPG |
| Variant (SEQ ID NO: | IFSSQPGHTPHPAASRDPVARTSPLQTPAAPGAAAGPALSPVPPVVH |
| 10) | LTLRQAVDDFSRRYRRDFAEMSSQLHLTPFTARGRFATVVEELFRDG |
| | VNWGRIVAFFEFGGVMCVESVNREMSPLVDNIALWMTEYLNRHLHTW |
| | IQDNGGWDAFVELYGPSMRPLFDFSWLSLKTLLSLALVGACITLGAY |
| | LGHK |

TABLE 4
Bcl-2 Variant Nucleotide Sequences

| | |
|---|---|
| Wild-type human | ATGGCTCACGCCGGCAGAACCGGCTACGACAACAGAGAAATCGTGAT |
| Bcl-2 (SEQ ID NO: 11) | GAAGTACATCCACTACAAGCTGAGCCAGCGGGGCTACGAATGGGATG |
| | CTGGCGACGTTGGAGCTGCTCCTCCTGGTGCTGCTCCTGCTCCTGGA |
| | ATCTTTAGCAGCCAGCCTGGCCACACACCTCATCCTGCCGCTTCTAG |
| | AGATCCCGTGGCCAGAACAAGCCCTCTGCAGACACCAGCTGCTCCAG |
| | GTGCTGCAGCTGGACCTGCACTTTCTCCAGTGCCTCCAGTGGTGCAC |
| | CTGACACTGAGACAGGCCGGCGACGACTTCAGCAGAAGATACAGACG |
| | GGACTTCGCCGAGATGTCCAGCCAGCTGCATCTGACCCCTTTCACAG |
| | CCAGAGGCAGATTCGCCACCGTGGTGGAAGAACTGTTCCGCGACGGT |
| | GTCAACTGGGGCAGAATCGTGGCCTTCTTCGAGTTCGGCGGCGTGAT |
| | GTGTGTGGAAAGCGTGAACCGGGAAATGAGCCCTCTGGTGGACAATA |
| | TCGCCCTGTGGATGACCGAGTACCTGAACCGGCATCTGCACACATGG |
| | ATCCAGGACAACGGCGGCTGGGATGCCTTCGTGGAACTGTACGGACC |
| | TAGCATGAGGCCCCTGTTCGACTTCTCTTGGCTGAGCCTGAAAACCC |
| | TGCTGTCTCTGGCCCTTGTGGGCGCCTGTATTACACTGGGAGCCTAC |
| | CTGGGCCACAAATGA |
| Human Bcl-2 G101V | ATGGCTCACGCCGGCAGAACCGGCTACGACAACAGAGAAATCGTGAT |
| Variant (SEQ ID NO: | GAAGTACATCCACTACAAGCTGAGCCAGCGGGGCTACGAATGGGATG |
| 12) | CTGGCGACGTTGGAGCTGCTCCTCCTGGTGCTGCTCCTGCTCCTGGA |
| | ATCTTTAGCAGCCAGCCTGGCCACACACCTCATCCTGCCGCTTCTAG |
| | AGATCCCGTGGCCAGAACAAGCCCTCTGCAGACACCAGCTGCTCCAG |
| | GTGCTGCAGCTGGACCTGCACTTTCTCCAGTGCCTCCAGTGGTGCAC |
| | CTGACACTGAGACAGGCCGTGGACGACTTCAGCCGGCGGTATAGAAG |
| | AGACTTCGCCGAGATGTCCAGCCAGCTGCATCTGACCCCTTTCACAG |
| | CCAGAGGCAGATTCGCCACCGTGGTGGAAGAACTGTTCCGCGACGGT |
| | GTCAACTGGGGCAGAATCGTGGCCTTCTTCGAGTTCGGCGGCGTGAT |
| | GTGTGTGGAAAGCGTGAACCGGGAAATGAGCCCTCTGGTGGACAATA |
| | TCGCCCTGTGGATGACCGAGTACCTGAACCGGCATCTGCACACATGG |
| | ATCCAGGACAACGGCGGCTGGGATGCCTTCGTGGAACTGTACGGACC |
| | TAGCATGAGGCCCCTGTTCGACTTCTCTTGGCTGAGCCTGAAAACCC |
| | TGCTGTCTCTGGCCCTTGTGGGCGCCTGTATTACACTGGGAGCCTAC |
| | CTGGGCCACAAATGA |

In some aspects, the payload comprises a MCL1 apoptosis regulator (MCL-1). MCL-1 has been shown to be important for the survival of HSCs and other hematopoietic progenitor cells. Bohler et al., Haematologica 106:3136-3148 (2021); and Chin et al., Front Cell Dev 9:704547(2021). Accordingly, in some aspects, using the squeeze processing methods provided herein, the expression of MCL-1 on the HSCs can be increased compared to a reference HSC. In some aspects, the expression of MCL-1 on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC.

In some aspects, the payload comprises Bcl-xL. In some aspects, using the squeeze processing methods provided herein, the expression of Bcl-xL on the HSCs can be increased compared to a reference HSC. In some aspects, the expression of Bcl-xL on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC.

In some aspects, the payload comprises Ced-9. In some aspects, using the squeeze processing methods provided herein, the expression of Ced-9 on the HSCs can be increased compared to a reference HSC. In some aspects, the expression of Ced-9 on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC.

In some aspects, the payload comprises a bfl-1. In some aspects, using the squeeze processing methods provided herein, the expression of bfl-1 on the HSCs can be increased compared to a reference HSC. In some aspects, the expression of bfl-1 on the HSCs is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold compared to the corresponding expression on the reference HSC.

In some aspects, a payload that is useful for the present disclosures comprises a cytokine or a growth factor, such as those that play a role in the engraftment of HSCs within the bone marrow of a subject. In some aspects, such payloads comprise a stem cell factor (SCF), a Fms-related tyrosine kinase 3 ligand (Flt3L), a thrombopoietin (TPO), or a combination thereof.

In some aspects, using the squeeze processing methods provided herein, HSCs can be modified to exhibit greater level of a cytokine or a growth factor described herein, wherein the greater expression allows the HSCs to better engraft within the bone marrow of a subject. In some aspects, compared to a reference HSC, the expression of SCF in the HSCs can be increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold. In some aspects, compared to a reference HSC, the expression of Flt3L can be increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold. In some aspects, compared to a reference HSC, the expression of TPO in the HSCs can be by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold.

In some aspects, a payload useful for the present disclosure comprises a cell adhesion molecule, such as those involved in the trafficking of HSCs to the bone marrow of a subject. For example, in some aspects, a cell adhesion molecule comprises an integrin, selectin, or both. Non-limiting examples of suitable integrins and selectins are known in the art and include, e.g., VLA-4, VLA-5, LFA-1, and combinations thereof. In some aspects, using the squeeze processing methods provided herein, the expression of a cell adhesion molecule on the HSCs can be increased compared to a reference HSC. In some aspects, compared to a reference HSC, the expression of a cell adhesion molecule can be increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold.

In some aspects, a payload that can be used with the present disclosure comprises a proliferative agent. As used herein, the term “proliferative agent” refers to any molecule that is capable of inducing the proliferation of a cell (e.g., HSCs). In some aspects, the proliferative agent induces the activation of a signaling pathway involved in regulating the proliferation of a cell, e.g., PI3K-ATK, Ras-ERK, or both. Accordingly, in some aspects, using the squeeze processing methods provided herein, the expression and/or activity of a proliferative agent can be increased in the HSCs compared to a reference HSC. In some aspects, the increased expression and/or activity of the proliferative agent is associated with increased proliferation of the HSCs. In some aspects, compared to a reference HSC, the proliferation of HSCs produced using the present disclosure is increased by at least about 2-fold, by at least about 3-fold, by at least about 4-fold, by at least about 5-fold, by at least about 6-fold, by at least about 7-fold, by at least about 8-fold, by at least about 9-fold, by at least about 10-fold, by at least about 15-fold, by at least about 20-fold, by at least about 25-fold, by at least about 30-fold, by at least about 40-fold, or by at least about 50-fold.

As described herein, in some aspects, a payload useful for the present disclosure comprises an agent that can regulate the expression and/or activity of the other payloads described herein (i.e., regulator). For example, G-protein-coupled receptor-associated sorting proteins 1 and 2 (GPRASP1/2) signaling has been described to inhibit CXCR4 expression. Knockdown of GPRASP1/2 has been shown to increase CXCR4 expression in HSCs. Morales-Hernandez et al., Blood 135:1111-1123 (2020); and Hernandez et al., Exp Hematol 64: S88 (2018). Accordingly, in some aspects, a payload useful for the present disclosure can reduce and/or inhibit the expression and/or activity of GPRASP1/2, such that the CXCR4 expression in the HSCs is increased. For example, in some aspects, the payload can comprise a siRNA (or any other gene editing tool) that selectively targets GPRASP1/2. Similarly, CD26 has also been shown to interfere with the expression and/or activity of CXCR4, and thereby, inducing the mobilization of HSCs. Fang et al., Stem Cell Res Ther 12(1): 17 (2021). Accordingly, in some aspects, a payload that can be used with the present disclosure is capable of reducing and/or inhibiting the expression and/or activity of CD26 (e.g., siRNA targeting CD26), such that the expression of CXCR4 is increased.

In addition to the above, payloads that can reduce or prevent the expression and/or activity of endogenous genes involved in HSC engraftment are also useful for the present disclosure. For example, such payloads can be used to decrease the expression of a homing receptor (e.g., CXCR4) naturally expressed on endogenous HSCs, such that the endogenous HSCs are mobilized and thus, allowing for greater engraftment of the therapeutic HSCs which have been modified to express higher levels of the homing receptor.

Any of the payloads described herein can be present in the cell suspension prior to, during, and/or after the passing step, in which the cell suspension is passed through the constriction. In some aspects, the cell suspension comprises at least about 2, at least about 3, at least about 4, at least about 5, at least about 6, at least about 7, at least about 8, at least about 9, or at least about 10 or more payloads. As further described elsewhere in the present disclosure, in some aspects, a cell suspension can be passed through multiple constrictions. In such aspects, a payload can be loaded into a cell when the cells pass through one or more of the multiple constrictions. In some aspects, a payload is loaded into a cell each time the cells pass through one or more of the multiple constrictions. When multiple payloads are involved, each of the payloads can be the same. In some aspects, one or more of the payloads are different.

Accordingly, as is apparent from the present disclosure, in some aspects, the squeeze processing methods described herein can be used to deliver multiple payloads to a cell. In some aspects, the multiple payloads can be delivered to a cell using a single squeeze processing (e.g., a cell suspension comprises the multiple payloads, which are delivered to the cell in combination; “concurrent delivery”). In some aspects, the multiple payloads can be delivered to a cell sequentially. As used herein, the term “sequential delivery” refers to the delivery of multiple payloads to a cell, where a first payload is delivered to the cell and then the second (or subsequent) payload is delivered to the cell. In some aspects, the first payload, the second payload, or both the first and second payloads can be delivered to the cell using squeeze processing. For instance, in some aspects, the first payload can be delivered to the cell using squeeze processing, and the second payload can be delivered to the cell using non-squeeze processing (e.g., transfection). In some aspects, the first payload can be delivered to the cell using non-squeeze processing (e.g., transfection), and the second payload can be delivered to the cell using squeeze processing. In some aspects, the first payload can be delivered to the cell using a first squeeze, and then the second payload can be delivered to the cell using a second squeeze (also referred to herein as “sequential squeeze” or “sequential squeeze processing”). Accordingly, sequential delivery useful for the present disclosure can comprise multiple squeeze processings. In some aspects, each of the multiple squeeze processings delivers a separate payload to the cell. In some aspects, one or more of the multiple squeeze processings do not involve the delivery of a payload. For instance, in some aspects, a sequential delivery method described herein comprises a first squeeze, a second squeeze, and a third squeeze, wherein the first squeeze comprises passing a cell without any payload through a first constriction, the second squeeze comprises passing the cell from the first squeeze through a second constriction to deliver a first payload to the cell, and the third squeeze comprises passing the cell from the second squeeze through a third constriction to deliver a second payload to the cell. Not to be bound by any one theory, in some aspects, passing the cell through the first constriction without any payload (i.e., the first squeeze) can help prepare the cell for subsequent payload deliveries, e.g., can improve the delivery efficiency of the first payload and/or the second payload.

In some aspects, the multiple payloads can be delivered to a cell repeatedly (e.g., at least two times, at least three times, at least four times, at least five times or more) using the squeeze processing methods described herein. As further described elsewhere in the present disclosure (see, e.g., section titled “Constrictions”), in some aspects, each of the multiple squeeze processing methods can be the same (e.g., same parameters). In some aspects, one or more of the multiple squeeze processing methods can be different (e.g., one or more delivery parameters described herein are different).

As is apparent from the present disclosure, a payload that is useful for the present disclosure is not particularly limited, as long as the payload is capable of modifying the HSCs, such that the HSCs exhibit one or more improved properties after the modification. Non-limiting examples of suitable payloads include a nucleic acid, a polypeptide, a lipid, a carbohydrate, a small molecule, a metal-containing compound, an antibody, a transcription factor, a nanoparticle, a liposome, a fluorescently tagged molecule, or combinations thereof. In some aspects, the nucleic acid comprises a DNA, RNA, or both. In some aspects, DNA comprises a recombinant DNA, a cDNA, a genomic DNA, or combinations thereof. In some aspects, RNA comprises a siRNA, a mRNA, a miRNA, a IncRNA, a tRNA, a shRNA, a self-amplifying mRNA (saRNA), a PNA, a LNA, or combinations thereof. In some aspects, the RNA is a mRNA.

As further described and demonstrated herein, a payload can be delivered using a squeeze processing method provided herein to a HSC alone or in combination (e.g., at least two, at least three, at least four, at least five, at least six, at least seven, at least about eight, at least about nine, or all of the listed exemplary transcription factors). In some aspects, where combinations of payloads are involved, they can be delivered to a HSC using a single squeeze processing (e.g., concurrent delivery). In some aspects, the combinations of payloads can be delivered to a HSC repeatedly. For instance, in some aspects, a combination of payloads is delivered to HSC with a first squeeze processing; then, the combination of payloads is delivered to the cells again with a second squeeze processing. In some aspects, the first squeeze processing includes a microfluidic device (e.g., chip) with multiple rows of constrictions, such that the squeeze process occurs on a single microfluidic device (e.g., chip). As further described herein, in some aspects, the second squeeze processing can occur immediately after the cells have gone through the first squeeze processing (e.g., immediately after the cells pass through the constriction of the first squeeze processing). In some aspects, the second squeeze processing can occur after some time after the first squeeze processing (e.g., at least about 1 minute, at least about 30 minutes, at least about 1 hour, at least about 6 hours, at least about 12 hours, or at least about 1 day after the cells pass through the constriction of the first squeeze processing).

While the present disclosure largely refers to the use of constrictions to deliver the payloads described herein to HSCs, it will be apparent to those skilled in the art that, in some aspects, the payloads can be delivered to the HSCs using any suitable delivery methods known in the art. Non-limiting examples of such delivery methods include: electroporation, vortex shedding forces (e.g., INDEE), lipid transfection, nanoparticle, sonoporation, and combinations thereof.

### III.C. Constrictions

### III.C.1. Microfluidic Channels

As described herein, a constriction is used to cause a physical deformity in the cells, such that perturbations are created within the cell membrane of the cells, allowing for the delivery of a payload into the cell. In some aspects, a constriction is within a channel contained within a microfluidic device (referred to herein as “microfluidic channel” or “channel”). Where multiple channels are involved, in some aspects, the multiple channels can be placed in parallel and/or in series within the microfluidic device. In some aspects, the cells described herein can be passed through at least about 2, at least about 3, at least about 4, at least about 5, at least about 6, at least about 7, at least about 8, at least about 9, at least about 10, at least about 20, at least about 30, at least about 40, at least about 50, at least about 75, at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1,000 or more separate constrictions. In some aspects, the cells described herein are passed through more than about 1,000 separate constrictions.

In some aspects, the multiple constrictions can be part of a single microfluidic device (e.g., multi-row constriction chip). In some aspects, one or more of the multiple constrictions can be part of different microfluidic devices. For instance, in some aspects, the cells described herein (e.g., HSCs) undergo a first squeeze processing, in which the cells pass through a first constriction in a first microfluidic device (e.g., chip). Then, after the cells have undergone the first squeeze processing (e.g., passed through the first constriction), the cells undergo a second squeeze processing, in which the cells pass through a second constriction in a second microfluidic device (e.g., chip). In some aspects, each of the constrictions are the same (e.g., has the same length, width, and/or depth). In some aspects, one or more of the constrictions are different. Where plurality of constrictions are used, the plurality of constrictions can comprise a first constriction which is associated with a first payload (e.g., mRNA encoding a CXCR4 or a variant thereof), and a second constriction which is associated with a second payload (e.g., mRNA encoding a Bcl-2 or a variant thereof), wherein the cell suspension passes through the first constriction such that the first payload is delivered to one or more cells of the plurality of cells, and then the cell suspension passes through the second constriction such that the second payload is delivered to the one or more cells of the plurality of cells. In some aspects, the cell suspension is passed through the second constriction at least about 1 minute, at least about 30 minutes, at least about 1 hour, at least about 6 hours, at least about 12 hours, or at least about 1 day after the cell suspension is passed through the first constriction.

In some aspects, where the cell suspension is passed through multiple constrictions (e.g., multiple squeeze processing), the cells remain viable after passing through each constriction. As is apparent from the present disclosure, in some aspects, multiple constrictions can comprise two or more constrictions present within a single microfluidic device (e.g., multi-row constriction chip), such the cells pass through the multiple constrictions sequentially. In some aspects, the multiple constrictions are part of separate microfluidic devices, such that a first constriction is associated with a first microfluidic device and a second constriction is associated with a second microfluidic device. For instance, as demonstrated herein, in some aspects, cells are passed through a first constriction (i.e., first squeeze processing), which is associated with a first microfluidic device (e.g., chip). After the cells have passed through the first constriction, the cells are passed through a second constriction (i.e., second squeeze processing), which is associated with a second microfluidic device (e.g., chip). In some aspects, after passing through the first constriction, the cells are cultured in a medium prior to passing the cells through the second constriction. In some aspects, the cells are cultured for at least about 1 minute, at least about 30 minutes, at least about 1 hour, at least about 6 hours, at least about 12 hours, or at least about 1 day before passing the cells through the second constriction. As is apparent from the present disclosure, in some aspects, the first and second constrictions have the same length, depth, and/or width. In some aspects, the first and second constrictions can have different length, depth, and/or width.

In some aspects, after passing through a constriction, at least about 50%, at least about 55%, at least about 60%, at least about 65%, at least about 70%, at least about 75%, at least about 80%, at least about 85%, at least about 90%, at least about 95%, or about 100% of the cells remain viable. Where the cells pass through multiple constrictions (e.g., part of a single microfluidic device or separate microfluidic devices), at least about 50%, at least about 55%, at least about 60%, at least about 65%, at least about 70%, at least about 75%, at least about 80%, at least about 85%, at least about 90%, at least about 95%, or about 100% of the cells remain viable after passing through each of the multiple constrictions. The viability of the cells can be measured using any suitable methods known in the art. In some aspects, the viability of the cells can be measured using a Nucleocounter NC-200, an Orflo Moxi Go II Cell Counter, or both.

Exemplary microfluidic channels containing cell-deforming constrictions for use in the methods disclosed herein are described in US Publ. No. 2020/0277566 A1, US Publ. No. 2020/0332243 A1, US Publ. No. 2020/0316604 A1, U.S. Provisional Appl. No. 63/131,423, and U.S. Provisional Appl. No. 63/131,430, each of which is incorporated herein by reference in its entirety.

In some aspects, a microfluidic channel described herein (i.e., comprising a constriction) includes a lumen and is configured such that a cell suspended in a buffer (e.g., cell suspension) can pass through the channel. Microfluidic channels useful for the present disclosure can be made using any suitable materials available in the art, including, but not limited to, silicon, metal (e.g., stainless steel), plastic (e.g., polystyrene), ceramics, glass, crystalline substrates, amorphous substrates, polymers (e.g., Poly-methyl methacrylate (PMMA), PDMS, Cyclic Olefin Copolymer (COC)), or combinations thereof. In some aspects, the material is silicon. Fabrication of the microfluidic channel can be performed by any method known in the art, including, but not limited to, dry etching for example deep reactive ion etching, wet etching, photolithography, injection molding, laser ablation, SU-8 masks, or combinations thereof. In some aspects, the fabrication is performed using dry etching.

In some aspects, a microfluidic channel useful for the present disclosure comprises an entrance portion, a center point, and an exit portion. In some aspects, the cross-section of one or more of the entrance portion, the center point, and/or the exit portion can vary. For example, the cross-section can be circular, elliptical, an elongated slit, square, hexagonal, or triangular in shape.

The entrance portion defines a constriction angle. In some aspects, by modulating (e.g., increasing or decreasing) the constriction angle, any clogging of the constriction can be reduced or prevented. In some aspects, the angle of the exit portion can also be modulated. For example, in some aspects, the angle of the exit portion can be configured to reduce the likelihood of turbulence that can result in non-laminar flow. In some aspects, the walls of the entrance portion and/or the exit portion are linear. In some aspects, the walls of the entrance portion and/or the exit portion are curved.

In some aspects, the length, depth, and/or width of the constriction can vary. In some aspects, by modulating (e.g., increasing or decreasing) the length, depth, and/or width of the constriction, the delivery efficiency of a payload can be regulated. As used herein, the term “delivery efficiency” refers to the amount of payload that is delivered into the cell. For instance, an increased delivery efficiency can occur when the total amount of payload that is delivered is increased.

In some aspects, the constriction has a length of less than about 1 μm. In some aspects the constriction has a length of about 0.1 μm to about 100 μm. In some aspects, the constriction has a length of less than about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, about 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the constriction has a length of about 0.5 μm. In some aspects, the constriction has a length of about 10 μm. In some aspects, the constriction has a length of about 70 μm.

In some aspects, the constriction has a depth of about 5 μm to about 90 μm. In some aspects, the constriction has a depth greater than or equal to about 5 μm, about 10 μm, about 20 μm, about 30 μm, about 40 μm, about 50 μm, about 60 μm, about 70 μm, about 80 μm, about 90 μm, about 100 μm, about 110 μm, or about 120 μm. In some aspects, the constriction has a depth of about 10 μm. In some aspects, the constriction has a depth of about 20 μm. In some aspects, the constriction has a depth of about 70 μm. In some aspects, the constriction has a depth of about 10 μm.

In some aspects, the constriction has a width of about 1 μm to about 10 μm. In some aspects, the constriction has a width of about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 3 μm, about 4 μm, about 4.5 μm, about 5 μm, about 6 μm, about 7 μm, about 8 μm, about 9 μm, or about 10 μm. In some aspects, the constriction has a width of about 6 μm. In some aspects, the constriction has a length of 10 μm, width of 6 μm, and a depth of 70 μm. In some aspects, the constriction has a width of about 3.5 μm. For example, in some aspects, the constriction that can used with the present disclosure has a depth of 10 μm, width of 3.5 μm, and a length of 70 μm.

In some aspects, the diameter of a constriction (e.g., contained within a microfluidic channel) is a function of the diameter of one or more cells that are passed through the constriction. Not to be bound by any one theory, in some aspects, the diameter of the constriction is less than that of the cells, such that a deforming force is applied to the cells as they pass through the constriction, resulting in the transient physical deformity of the cells.

Accordingly, in some aspects, the diameter of the constriction (also referred to herein as “constriction size”) is about 20% to about 99% of the diameter of the cell. In some aspects, the constriction size is about 20%, about 30%, about 40%, about 50%, about 60%, about 70%, about 80%, about 90%, or about 99% of the cell diameter. As is apparent from the present disclosure, by modulating (e.g., increasing or decreasing) the diameter of a constriction, the delivery efficiency of a payload into a cell can also be regulated.

### III.C.2. Surfaces Having Pores

In some aspects, a constriction described herein comprises a pore, which is contained in a surface. Non-limiting examples of pores contained in a surface that can be used with the present disclosure are described in, e.g., US Publ. No. 2019/0382796 A1, which is incorporated herein by reference in its entirety.

In some aspects, a surface useful for the present disclosure (i.e., comprising one or more pores that can cause a physical deformity in a cell as it passes through the pore) can be made using any suitable materials available in the art and/or take any one of a number of forms. Non-limiting examples of such materials include synthetic or natural polymers, polycarbonate, silicon, glass, metal, alloy, cellulose nitrate, silver, cellulose acetate, nylon, polyester, polyethersulfone, polyacrylonitrile (PAN), polypropylene, PVDF, polytetrafluorethylene, mixed cellulose ester, porcelain, ceramic, or combinations thereof.

In some aspects, the surface comprises a filter. In some aspects, the filter is a tangential flow filter. In some aspects, the surface comprises a membrane. In some aspects, the surface comprises a sponge or sponge-like matrix. In some aspects, the surface comprises a matrix. In some aspects, the surface comprises a tortuous path surface. In some aspects, the tortuous path surface comprises cellulose acetate.

The surface disclosed herein (i.e., comprising one or more pores) can have any suitable shape known in the art. Where the surface has a 2-dimensional shape, the surface can be, without limitation, circular, elliptical, round, square, star-shaped, triangular, polygonal, pentagonal, hexagonal, heptagonal, or octagonal. In some aspects, the surface is round in shape. Where the surface has a 3-dimensional shape, in some aspects, the surface can be, without limitation, cylindrical, conical, or cuboidal.

As is apparent from the present disclosure, a surface that is useful for the present disclosure (e.g., comprising one or more pores) can have various cross-sectional widths and thicknesses. In some aspects, the cross-sectional width of the surface is between about 1 mm and about 1 m. In some aspects, the surface has a defined thickness. In some aspects, the surface thickness is uniform. In some aspects, the surface thickness is variable. For example, in some aspects, certain portions of the surface are thicker or thinner than other portions of the surface. In such aspects, the thickness of the different portions of the surface can vary by about 1% to about 90%. In some aspects, the surface is between about 0.01 μm to about 5 mm in thickness.

The cross-sectional width of the pores can depend on the type of cell that is being targeted with a payload. In some aspects, the pore size is a function of the diameter of the cell of cluster of cells to be targeted. In some aspects, the pore size is such that a cell is perturbed (i.e., physically deformed) upon passing through the pore. In some aspects, the pore size is less than the diameter of the cell. In some aspects, the pore size is about 20% to about 99% of the diameter of the cell. In some aspects, the pore size is about 20%, about 30%, about 40%, about 50%, about 60%, about 70%, about 80%, about 90%, or about 99% of the diameter of the cell. In some aspects, the pore size is about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about μm, about 9 μm, about 10 μm, about 11 μm, about 12 μm, about 13 μm, about 14 μm, or about 15 μm or more.

The entrances and exits of a pore can have a variety of angles. In some aspects, by modulating (e.g., increasing or decreasing) the pore angle, any clogging of the pore can be reduced or prevented. In some aspects, the flow rate (i.e., the rate at which a cell or a suspension comprising the cell passes through the pore) is between about 0.001 mL/cm/sec to about 100 L/cm/sec. For example, the angle of the entrance or exit portion can be between about 0 and about 90 degrees. In some aspects, the pores have identical entrance and exit angles. In some aspects, the pores have different entrance and exit angles. In some aspects, the pore edge is smooth, e.g., rounded or curved. As used herein, a “smooth” pore edge has a continuous, flat, and even surface without bumps, ridges, or uneven parts. In some aspects, the pore edge is sharp. As used herein, a “sharp” pore edge has a thin edge that is pointed or at an acute angle. In some aspects, the pore passage is straight. As used herein, a “straight” pore passage does not contain curves, bends, angles, or other irregularities. In some aspects, the pore passage is curved. As used herein, a “curved” pore passage is bent or deviates from a straight line. In some aspects, the pore passage has multiple curves, e.g., about 2, about 3, about 4, about 5, about 6, about 7, about 8, about 9, about 10 or more curves.

The pores can have any shape known in the art, including a 2-dimensional or 3-dimensional shape. The pore shape (e.g., the cross-sectional shape) can be, without limitation, circular, elliptical, round, square, star-shaped, triangular, polygonal, pentagonal, hexagonal, heptagonal, and octagonal. In some aspects, the cross-section of the pore is round in shape. In some aspects, the 3-dimensional shape of the pore is cylindrical or conical. In some aspects, the pore has a fluted entrance and exit shape. In some aspects, the pore shape is homogenous (i.e., consistent or regular) among pores within a given surface. In some aspects, the pore shape is heterogeneous (i.e., mixed or varied) among pores within a given surface.

A surface useful for the present disclosure can have a single pore. In some aspects, a surface useful for the present disclosure comprises multiple pores. In some aspects, the pores encompass about 10% to about 80% of the total surface area of the surface. In some aspects, the surface contains about 1.0×10⁵ to about 1.0×10³⁰ total pores. In some aspects, the surface comprises between about 10 and about 1.0×10¹⁵ pores per mm² surface area.

The pores can be distributed in numerous ways within a given surface. In some aspects, the pores are distributed in parallel within a given surface. In some aspects, the pores are distributed side-by-side in the same direction and are the same distance apart within a given surface. In some aspects, the distribution of the pores is ordered or homogeneous. In such aspects, the pores can be distributed in a regular, systematic pattern, or can be the same distance apart within a given surface. In some aspects, the distribution of the pores is random or heterogeneous. For instance, in some aspects, the pores are distributed in an irregular, disordered pattern, or are different distances apart within a given surface.

In some aspects, multiple surfaces are used, such that a cell passes through multiple pores, wherein the pores are on different surfaces. In some aspects, multiple surfaces are distributed in series. The multiple surfaces can be homogeneous or heterogeneous in surface size, shape, and/or roughness. The multiple surfaces can further contain pores with homogeneous or heterogeneous pore size, shape, and/or number, thereby enabling the simultaneous delivery of a range of payloads into different cell types.

In some aspects, an individual pore, e.g., of a surface that can be used with the present disclosure, has a uniform width dimension (i.e., constant width along the length of the pore passage). In some aspects, an individual pore has a variable width (i.e., increasing or decreasing width along the length of the pore passage). In some aspects, pores within a given surface have the same individual pore depths. In some aspects, pores within a given surface have different individual pore depths. In some aspects, the pores are immediately adjacent to each other. In some aspects, the pores are separated from each other by a distance. In some aspects, the pores are separated from each other by a distance of about 0.001 μm to about 30 mm.

In some aspects, the surface is coated with a material. The material can be selected from any material known in the art, including, without limitation, Teflon, an adhesive coating, surfactants, proteins, adhesion molecules, antibodies, anticoagulants, factors that modulate cellular function, nucleic acids, lipids, carbohydrates, transmembrane proteins, or combinations thereof. In some aspects, the surface is coated with polyvinylpyrrolidone. In some aspects, the material is covalently attached to the surface. In some aspects, the material is non-covalently attached to the surface. In some aspects, the surface molecules are released at the cells pass through the pores.

In some aspects, the surface has modified chemical properties. In some aspects, the surface is hydrophilic. In some aspects, the surface is hydrophobic. In some aspects, the surface is charged. In some aspects, the surface is positively and/or negatively charged. In some aspects, the surface can be positively charged in some regions and negatively charged in other regions. In some aspects, the surface has an overall positive or overall negative charge. In some aspects, the surface can be any one of smooth, electropolished, rough, or plasma treated. In some aspects, the surface comprises a zwitterion or dipolar compound. In some aspects, the surface is plasma treated.

In some aspects, the surface is contained within a larger module. In some aspects, the surface is contained within a syringe, such as a plastic or glass syringe. In some aspects, the surface is contained within a plastic filter holder. In some aspects, the surface is contained within a pipette tip.

### III.D. Cell Perturbation

As described herein, as a cell passes through a constriction, it becomes physically deformed, such that there is a perturbation (e.g., a hole, tear, cavity, aperture, pore, break, gap, perforation) in the cell membrane of the cell. Such perturbation in the cell membrane is temporary and sufficient for any of the payloads described herein to be delivered into the cell. Cells have self-repair mechanisms that allow the cells to repair any disruption in their cell membrane. See Blazek et al., Physiology (Bethesda) 30(6): 438-48 (November 2015), which is incorporated herein by reference in its entirety. Accordingly, in some aspects, once the cells have passed through the constriction (e.g., microfluidic channel or pores), the perturbations in the cell membrane can be reduced or eliminated, such that the payload that was delivered into the cell does not exit the cell.

In some aspects, the perturbation in the cell membrane lasts from about 1.0×10⁻⁹ seconds to about 2 hours after the pressure is removed (e.g., cells have passed through the constriction). In some aspects, the cell perturbation lasts for about 1.0×10⁻⁹ second to about 1 second, for about 1 second to about 1 minute, or for about 1 minute to about 1 hour. In some aspects, the cell perturbation lasts for between about 1.0×10⁻⁹ second to about 1.0×10⁻¹ second, between about 1.0×10⁻⁹ second to about 1.0×10⁻² second, between about 1.0×10⁻⁹ second to about 1.0×10⁻³ second, between about 1.0×10⁻⁹ second to about 1.0×10⁻⁴ second, between about 1.0×10⁻⁹ second to about 1.0×10⁻⁵ second, between about 1.0×10⁻⁹ second to about 1.0×10⁻⁶ second, between about 1.0×10⁻⁹ second to about 1.0×10⁻⁷ second, or between about 1.0×10⁻⁹ second to about 1.0×10⁻⁸ second. In some aspects, the cell perturbation lasts for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. The cell perturbations (e.g., pores or holes) created by the methods described herein are not formed as a result of assembly of polypeptide subunits to form a multimeric pore structure such as that created by complement or bacterial hemolysins.

In some aspects, as the cell passes through the constriction, the pressure applied to the cells temporarily imparts injury to the cell membrane that causes passive diffusion of material through the perturbation. In some aspects, the cell is only deformed or perturbed for a brief period of time, e.g., on the order of 100 μs or less to minimize the chance of activating apoptotic pathways through cell signaling mechanisms, although other durations are possible (e.g., ranging from nanoseconds to hours). In some aspects, the cell is deformed for less than about 1.0×10⁻⁹ second to less than about 2 hours. In some aspects, the cell is deformed for less than about 1.0×10⁻⁹ second to less than about 1 second, less than about 1 second to less than about 1 minute, or less than about 1 minute to less than about 1 hour. In some aspects, the cell is deformed for about 1.0×10⁻⁹ second to about 2 hours. In some aspects, the cell is deformed for about 1.0×10⁻⁹ second to about 1 second, about 1 second to about 1 minute, or about 1 minute to about 1 hour. In some aspects, the cell is deformed for between any one of about 1.0×10⁻⁹ second to about 1.0×10⁻¹ second, about 1.0×10⁻⁹ second to about 1.0×10⁻² second, about 1.0×10⁻⁹ second to about 1.0×10⁻³ second, about 1.0×10⁻⁹ second to about 1.0×10⁻⁴ second, about 1.0×10⁻⁹ second to about 1.0×10⁻⁵ second, about 1.0×10⁻⁹ second to about 1.0×10⁻⁶ second, about 1.0×10⁻⁹ second to about 1.0×10⁻⁷ second, or about 1.0×10⁻⁹ second to about 1.0×10⁻⁸ second. In some aspects, the cell is deformed or perturbed for about 1.0×10⁻⁸ second to about 1.0×10⁻¹ second, for about 1.0×10⁻⁷ second to about 1.0×10⁻¹ second, about 1.0×10⁻⁶ second to about 1.0×10⁻¹ second, about 1.0×10⁻⁵ second to about 1.0×10⁻¹ second, about 1.0×10⁻⁴ second to about 1.0×10⁻¹ second, about 1.0×10⁻³ second to about 1.0×10⁻¹ second, or about 1.0×10⁻² second to about 1.0×10⁻¹ second. In some aspects, deforming the cell includes deforming the cell for a time ranging from, without limitation, about 1 μs to at least about 750 μs, e.g., at least about 1 μs, at least about 10 μs, at least about 50μs, at least about 100 μs, at least about 500 μs, or at least about 750 μs.

In some aspects, the delivery of a payload into the cell occurs simultaneously with the cell passing through the constriction. In some aspects, delivery of the payload into the cell can occur after the cell passes through the constriction (i.e., when perturbation of the cell membrane is still present and prior to cell membrane of the cells being restored). In some aspects, delivery of the payload into the cell occurs on the order of minutes after the cell passes through the constriction. In some aspects, a perturbation in the cell after it passes through the constriction is corrected within the order of about five minutes after the cell passes through the constriction.

In some aspects, the viability of a cell (e.g., stem cell or PBMC) after passing through a constriction is about 5% to about 100%. In some aspects, the cell viability after passing through the constriction is at least about 5%, at least about 10%, at least about 20%, at least about 30%, at least about 40%, at least about 50%, at least about 60%, at least about 70%, at least about 75%, at least about 80%, at least about 85%, at least about 90%, at least about 95%, or at least about 99%. In some aspects, the cell viability is measured from about 1.0×10⁻² second to at least about 10 days after the cell passes through the constriction. For example, the cell viability can be measured from about 1.0×10⁻² second to about 1 second, about 1 second to about 1 minute, about 1 minute to about 30 minutes, or about 30 minutes to about 2 hours after the cell passes through the constriction. In some aspects, the cell viability is measured about 1.0×10⁻² second to about 2 hours, about 1.0×10⁻² second to about 1 hour, about 1.0×10⁻² second to about 30 minutes, about 11.0×10⁻² second to about 1 minute, about 1.0×10⁻² second to about 30 seconds, about 1.0×10⁻² second to about 1 second, or about 1.0×10⁻² second to about 0.1 second after the cell passes through the constriction. In some aspects, the cell viability is measured about 1.5 hours to about 2 hours, about 1 hour to about 2 hours, about 30 minutes to about 2 hours, about 15 minutes to about 2 hours, about 1 minute to about 2 hours, about 30 seconds to about 2 hours, or about 1 second to about 2 hours after the cell passes through the constriction. In some aspects, the cell viability is measured about 2 hours to about 5 hours, about 5 hours to about 12 hours, about 12 hours to about 24 hours, or about 24 hours to about 10 days after the cell passes through the constriction.

### III.E. Delivery Parameters

As is apparent from the present disclosure, a number of parameters can influence the delivery efficiency of a payload into a cell using the squeeze processing methods provided herein. Accordingly, by modulating (e.g., increasing or decreasing) one or more of the delivery parameters, the delivery of a payload into a cell can be improved. Therefore, in some aspects, the present disclosure relates to a method of increasing the delivery of a payload into a cell, wherein the method comprises modulating one or more parameters under which a cell suspension is passed through a constriction, wherein the cell suspension comprises a population of the cells, and wherein the one or more parameters increase the delivery of a payload into one or more cells of the population of cells compared to a reference parameter. As described elsewhere in the present disclosure, the payload can be in contact with the population of cells before, during, or after the squeezing step.

In some aspects, by modulating one or more of the delivery parameters, the delivery of the payload into the one or more cells is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold, compared to a delivery of the payload agent into a corresponding cell using the reference parameter.

In some aspects, the one or more delivery parameters that can be modulated to increase the delivery efficiency of a parameter comprises a cell density (i.e., the concentration of the cells present, e.g., in the cell suspension), pressure, or both. Additional examples of delivery parameters that can be modulated are provided elsewhere in the present disclosure.

In some aspects, the cell density is about 1×10⁵ cells/mL, about 2×10⁵ cells/mL, about 3×10⁵ cells/mL, about 4×10⁵ cells/mL, about 5×10⁵ cells/mL, about 6×10⁵ cells/mL, about 7×10⁵ cells/mL, about 8×10⁵ cells/mL, about 9×10⁵ cells/mL, about 1×10⁶ cells/mL, about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 1×10⁷ cells/mL, about 2×10⁷ cells/mL, about 3×10⁷ cells/mL, about 4×10⁷ cells/mL, about 5×10⁷ cells/mL, about 6×10⁷ cells/mL, about 7×10⁷ cells/mL, about 8×10⁷ cells/mL, about 9×10⁷ cells/mL, about 1×10⁸ cells/mL, about 1.1×10⁸ cells/mL, about 1.2×10⁸ cells/mL, about 1.3×10⁸ cells/mL, about 1.4 x 108 cells/mL, about 1.5 x 108 cells/mL, about 2.0 x 108 cells/mL, about 3.0×10⁸ cells/mL, about 4.0×10⁸ cells/mL, about 5.0×10⁸ cells/mL, about 6.0×10⁸ cells/mL, about 7.0×10⁸ cells/mL, about 8.0×10⁸ cells/mL, about 9.0×10⁸ cells/mL, or about 1.0×10⁹ cells/mL or more. In some aspects, the cell density is between about 6×10⁷ cells/mL and about 1.2×10⁸ cells/mL.

In some aspects, the pressure is about 20 psi, about 25 psi, about 30 psi, about 35 psi, about 40 psi, about 50 psi, about 55 psi, about 60 psi, about 65 psi, about 70 psi, about 75 psi, about 80 psi, about 85 psi, about 90 psi, about 95 psi, about 100 psi, about 110 psi, about 120 psi, about 130 psi, about 140 psi, about 150 psi, about 160 psi, about 170 psi, about 180 psi, about 190 psi, or about 200 psi or more. In some aspects, the pressure is between about 30 psi and about 90 psi. In some aspects, the pressure is about 20 psi.

In some aspects, the particular type of device (e.g., microfluidic chip) can also have an effect on the delivery efficiency of a payload described herein. In the case of a microfluidic chip, different chips can have different constriction parameters, e.g., length, depth, and width of the constriction; entrance angle, exit angle, length, depth, and width of the approach region, etc. As described herein, such variables can influence the delivery of a payload into a cell using the squeeze processing methods of the present disclosure.

In some aspects, the length of the constriction is up to 100 μm. For instance, in some aspects, the length is about 0 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2.5 μm, about 5 μm, about 7.5 μm, 10 μm, about 12.5 μm, about 15 μm, about 20 μm, about 30 μm, about 40 μm, about 50 μm, about 60 μm, about 70 μm, about 80 μm, about 90 μm, or about 100 μm. In some aspects, the length of the constriction is less than 1 μm. In some aspects, the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm. In some aspects, the constriction has a length of about 10 μm. In some aspects, the constriction has a length of about 0.5 μm. In some aspects, the constriction has a length of about 70 μm. In some aspects, the constriction has a length of about 0 μm. For example, in some aspects, a microfluidic device (e.g., chip) useful for the present disclosure comprises a constriction that resembles two points of a diamond coming together, such that the length of the constriction is about 0 μm.

In some aspects, the width of the constriction is up to about 10 μm. In some aspects, the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm. In some aspects, the width is between about 1 μm to about 10 μm. In some aspects, the width is about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 3 μm, about 4 μm, about 5 μm, about 6 μm, about 7 μm, about 8 μm, about 9 μm, or about 10 μm. In some aspects, the width of the constriction is about 6 μm. In some aspects, the constriction has a width of about 3.5 μm.

In some aspects, the depth of the constriction is at least about 1 μm. In some aspects, the depth of the constriction is at least about 1 μm, at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm. In some aspects, the depth is between about 5 μm to about 90 μm. In some aspects, the depth is about 5 μm, about 10 μm, about 15 μm, about 20 μm, about 30 μm, about 40 μm, about 50 μm, about 60 μm, about 70 μm, about 80 μm, or about 90 μm. In some aspects, the constriction has a depth of about 20 μm. In some aspects, the depth of the constriction is about 70 μm. In some aspects, the constriction has a depth of about 10 μm.

In some aspects, the depth is about 10 μm, the width is about 3.5 μm, and length is about 70 μm, and the pressure is about 20 psi.

In some aspects, a constriction useful for the present disclosure comprises a width and a depth, wherein the width of the constriction is any distance described herein, and wherein the depth of the constriction is any distance described herein. In some aspects, the constriction comprises a width and a depth, wherein the width of the constriction is about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 3 μm, about 4 μm, about 5 μm, about 6 μm, about 7 μm, about 8 μm, about 9 μm, or about 10 μm, and wherein the depth of the constriction is about 5 μm, about 10 μm, about 20 μm, about 30 μm, about 40 μm, about 50 μm, about 60 μm, about 70 μm, about 80 μm, or about 90 μm. In some aspects, such a constriction can comprise a length of about 0 μm (e.g., a constriction that resembles two points of a diamond coming together, such that the length of the constriction is about 0 μm).

Additional examples of parameters that can influence the delivery of a payload into the cell include, but are not limited to, the dimensions of the constriction (e.g., length, width, and/or depth), the entrance angle of the constriction, the surface properties of the constrictions (e.g., roughness, chemical modification, hydrophilic, hydrophobic), the operating flow speeds, payload concentration, the amount of time that the cell recovers, or combinations thereof. Further parameters that can influence the delivery efficiency of a payload can include the velocity of the cell in the constriction, the shear rate in the constriction, the viscosity of the cell suspension, the velocity component that is perpendicular to flow velocity, and time in the constriction. Such parameters can be designed to control delivery of the payload.

In some aspects, the temperature used in the methods of the present disclosure can also have an effect on the delivery efficiency of the payloads into the cell, as well as the viability of the cell. In some aspects, the squeeze processing method is performed between about −5° C. and about 45° C. For example, the methods can be carried out at room temperature (e.g., about 20° C.), physiological temperature (e.g., about 37° C.), higher than physiological temperature (e.g., greater than about 37° C. to 45° C. or more), or reduced temperature (e.g., about −5° C. to about 4° C.), or temperatures between these exemplary temperatures.

Various methods can be utilized to drive the cells through the constrictions. For example, pressure can be applied by a pump on the entrance side (e.g., gas cylinder, or compressor), a vacuum can be applied by a vacuum pump on the exit side, capillary action can be applied through a tube, and/or the system can be gravity fed. Displacement based flow systems can also be used (e.g., syringe pump, peristaltic pump, manual syringe or pipette, pistons, etc.). In some aspects, the cells are passed through the constrictions by positive pressure. In some aspects, the cells are passed through the constrictions by constant pressure or variable pressure. In some aspects, pressure is applied using a syringe. In some aspects, pressure is applied using a pump. In some aspects, the pump is a peristaltic pump or a diaphragm pump. In some aspects, pressure is applied using a vacuum. In some aspects, the cells are passed through the constrictions by g-force. In some aspects, the cells are passed through the constrictions by capillary pressure.

In some aspects, fluid flow directs the cells through the constrictions. In some aspects, the fluid flow is turbulent flow prior to the cells passing through the constriction. Turbulent flow is a fluid flow in which the velocity at a given point varies erratically in magnitude and direction. In some aspects, the fluid flow through the constriction is laminar flow. Laminar flow involves uninterrupted flow in a fluid near a solid boundary in which the direction of flow at every point remains constant. In some aspects, the fluid flow is turbulent flow after the cells pass through the constriction. The velocity at which the cells pass through the constrictions can be varied. In some aspects, the cells pass through the constrictions at a uniform cell speed. In some aspects, the cells pass through the constrictions at a fluctuating cell speed.

In some aspects, a combination treatment is used to deliver a payload, e.g., the methods described herein followed by exposure to an electric field downstream of the constriction. In some aspects, the cell is passed through an electric field generated by at least one electrode after passing through the constriction. In some aspects, the electric field assists in delivery of a payload to a second location inside the cell such as the cell nucleus. In some aspects, one or more electrodes are in proximity to the cell-deforming constriction to generate an electric field. In some aspects, the electric field is between about 0.1 kV/m to about 100 MV/m. In some aspects, an integrated circuit is used to provide an electrical signal to drive the electrodes. In some aspects, the cells are exposed to the electric field for a pulse width of between about 1 ns to about 1 s and a period of between about 100 ns to about 10 s.

### III.F. Therapeutic Uses

In some aspects, the present disclosure relates to the use of the modified HSCs produced using the squeeze processing methods described herein to treat various diseases or disorders. As is apparent from the present disclosure, the methods and compositions provided herein can be useful for diseases and disorders where cell replacement therapies can be used as a treatment. By replacing cells that are damaged with the cells produced using the methods provided herein, in some aspects, one or more functions associated with the damaged cells can be restored, and thereby, treat the disease or disorder. For instance, in some aspects, the modified HSCs that are produced using the squeeze processing methods provided herein could be administered to a subject suffering from a blood disorder (e.g., hemoglobinopathies). The administration of such HSCs could be useful in improving one or more symptoms associated with the blood disorder. Non-limiting examples of blood disorders that can be treated using the methods provided herein comprise Sickle Cell Disease (SCD), Thalassemia Syndrome, severe aplastic anemia, Fanconi anemia, Paroxysmal nocturnal hemoglobinuria, Pure red cell aplasia, congenital amegakaryocytic thrombocytopenia, or a combination thereof. In some aspects, a disease or disorder that can be treated with the present disclosure comprises an immune disorder. Non-limiting examples of immune disorders include Severe combined immunodeficiency, Wiskott-Aldrich syndrome, or both. In some aspects, a disease or disorder that can be treated with the present disclosure comprises a metabolic disorder. Non-limiting examples of metabolic disorders include Krabbe disease (GLD), Hurler syndrome, Adrenoleukodystrophy, Metachromatic leukodystrophy, Fabry disease, Gaucher disease, cystinosis, Hunter syndrome, Pompe disease, or a combination thereof.

As is apparent from the present disclosure, compared to other HSC-based treatments available in the art, the therapeutic methods provided herein can be used without the need to precondition a subject to be treated (e.g., with a toxic myeloablative therapy, e.g., busulfan administration). Non-limiting examples of such myelolablative preconditioning regimen includes: an irradiation, a chemotherapy, a small molecule, an antibody, or a combination thereof. As described herein, through the use of mobilization factors (e.g., plerixaflor) and/or apoptotic factors (e.g., venetoclax) which are not associated with any harsh side effects, the therapeutic methods provided herein allow for much safer and efficient approach to promoting the engraftment of therapeutically modified HSCs.

### IV. Compositions of the Disclosures

In some aspects, the disclosure provides a system for delivery of a payload into a cell (e.g., HSC), the system comprising a microfluidic channel described herein, a cell suspension comprising a plurality of the cells and the payload; wherein the constriction is configured such that the plurality of cells can pass through the microfluidic channel, wherein the passing of the plurality of cells causes a deformity and disruption of the cell membrane of the cell, allowing the payload to enter the cell.

In some aspects, the disclosure provides a system for delivering a payload, the system comprising a surface with pores, a cell suspension comprising a plurality of the cells and the payload; wherein the surface with pores is configured such that the plurality of cells can pass through the pores, wherein the passing of the plurality of cells causes a deformity and disruption of the cell membrane of the cell, allowing the payload to enter the cell. In some aspects, the surface is a filter or a membrane. In some aspects of the above aspects, the system further comprises at least one electrode to generate an electric field. In some aspects, the system is used to deliver a payload into a cell by any of the methods described herein. The system can include any aspect described for the methods disclosed above, including microfluidic channels or a surface having pores to provide cell-deforming constrictions, cell suspensions, cell perturbations, delivery parameters. In some aspects, the delivery parameters, such as operating flow speeds, cell and compound concentration, velocity of the cell in the constriction, and the composition of the cell suspension (e.g., osmolarity, salt concentration, serum content, cell concentration, pH, etc.) are optimized for delivery of a payload into the cell.

In some aspects, the disclosure provides a cell produced using any of the methods provided herein (e.g., HSC). In some aspects, provided herein is a cell comprising a perturbation in the cell membrane, wherein the perturbation is due to one or more parameters which deform the cell (e.g., delivery parameters described herein), thereby creating the perturbation in the cell membrane of the cell such that a payload can enter the cell. In some aspects, provided herein is a cell comprising a payload, wherein the payload entered the cell through a perturbation in the cell membrane, which was due to one or more parameters which deform the cell (e.g., delivery parameters described herein) and thereby creating the perturbation in the cell membrane of the cell such that the payload entered the cell. In some aspects, such cells can comprise a modified HSC described herein (e.g., exhibiting increased resistance to mobilization and/or apoptotic factors).

In some aspects, the present disclosure provides a composition comprising a plurality of cells, wherein the plurality of cells were produced by any of the methods provided herein. Also provided herein is a composition comprising a population of cells and a payload under one or more parameters, which result in deformation of one or more cells of the population of cells and thereby creating perturbations in the cell membrane of the one or more cells, and wherein the perturbations in the cell membrane allows the payload to enter the one or more cells.

In some aspects, the present disclosure further provides a composition comprising a population of modified hematopoietic stem cells (HSCs), wherein the modified HSCs comprise a payload, which is capable of: (i) increasing the resistance of the modified HSCs to a mobilization factor, (ii) increasing the resistance of the modified HSCs to an inhibitor of an anti-apoptotic factor, (iii) increasing the resistance of the modified HSCs to a depleting factor, (iv) increasing the expression of a homing factor on the modified HSCs, or (v) a combination thereof. As further described elsewhere in the present disclosure, in some aspects, the payload is delivered to the HSCs using a method other than the squeeze processing delivery methods provided herein. Non-limiting examples of such methods are provided elsewhere in the present disclosure.

Also provided are kits or articles of manufacture for use in delivering into a cell a payload as described herein. In some aspects, the kits comprise the compositions described herein (e.g. a microfluidic channel or surface containing pores, cell suspensions, and/or payload) in suitable packaging. Suitable packaging materials are known in the art, and include, for example, vials (such as sealed vials), vessels, ampules, bottles, jars, flexible packaging (e.g., sealed Mylar or plastic bags), and the like. These articles of manufacture can further be sterilized and/or sealed.

The present disclosure also provides kits comprising components of the methods described herein and can further comprise instruction(s) for performing said methods to deliver a payload into a cell. The kits described herein can further include other materials, including other buffers, diluents, filters, needles, syringes, and package inserts with instructions for performing any methods described herein; e.g., instructions for delivering a payload into a cell.

The following examples are offered by way of illustration and not by way of limitation.

### EXAMPLES

### Example 1: Analysis of the Effect of Squeeze Processing on HSC Engraftment Efficiency

To assess any potential impact on engraftment efficiency, CD34+ HSCs were loaded with mRNA encoding GFP using the squeeze processing methods provided herein and adoptively transferred into NSG and NBSGW recipient mice. The specific methods used are provided below.

### Squeeze Processing

Two 5 M (i.e., 5×10⁶) cell vials were thawed and diluted with FACS buffer. Cells were centrifuged at 400 rcf at room temperature to 2 M/ml. Cells were transferred to a 12 well non-treated plate and cultured overnight in complete CD34+ media at 37° C. The supernatant was aspirated, and cells were resuspended with 3 ml of CD34+ media. The cells were diluted to 2 M/ml, plated, and cultured overnight at 37° C. The cells were prepared for squeeze processing. Table 5 provides a description of the different test groups and the specific cell suspension solutions used for the squeeze processing.

TABLE 5
Squeeze Processing Cell Suspension Solutions

| | |
|---|---|
| Group A NSG Mice | 250 μL OptiMEM + of 250 μL 2X HSCs |
| Group B NSG Mice | 300 μL OptiMEM-mRNA master mix + 300 |
| | μL 2X HSCs |
| Group C NBSGW Mice | 250 μL OptiMEM + of 250 μL 2X HSCs |
| Group D NBSGW Mice | 300 μL OptiMEM-mRNA master mix + 300 |
| | μL 2X HSCs |

As shown in Table 5, Groups A and C were squeeze processed at room temperature with no mRNA using a microfluidic constriction (10 μm depth, 3.5 μm width, and 70 μm length) at 20 psi. Cells were then transferred into a 15 ml conical containing 5 mL CD34+ media. Groups B and D were squeezed processed with GFM mRNA at room temperature using a microfluidic constriction (10 μm depth, 3.5 μm width, and 70 μm length) at 20 psi. Cells were transferred into a 15 ml conical containing 5 mL CD34+ media. Cells from the different groups were centrifuged separately, at 500 rcf for 5 minutes at room temperature. The supernatant was aspirated and the cells resuspended in 1 mL of sterile PBS.

### Adoptive Transfer

All mice underwent tail vein injections. Each mouse received 100 ul injection containing 250 K HSC cells according to the groups specified above.

### Bone Marrow Harvest and Analysis

Six weeks after injection, bone marrow was harvested from the femur and tibia of each mice for all mice. The tibia and femur were cut close to the knee joint and placed in a microcentrifuge tube. 25 μL FACs buffer was added to an Eppendorf tube and the microcentrifuge tube containing the sample was placed inside. The samples were centrifuged at 10,000 rcf for 15 seconds. . . . The cells were then treated with 2 ml of ACK lysis buffer for 1-2 min to remove red blood cells. Following lysis, cells were washed and resuspended using FACs buffer.

### Spleen Harvest and Analysis

Six weeks after injection, spleens were collected from all mice and placed each into Eppendorf tubes containing 1 ml FACs buffer. Each spleen sample is smashed through a 70 μm filter into a 50 ml conical. Samples are rinsed with 10 ml FACs buffer. The samples were spun at 400 rcf for 5 minutes, then resuspended in 2 ml ACK buffer. The samples were incubated for 1-2 minutes and then quenched with 10 ml FACs buffer. The samples were then filtered through a 70um filter, washed, then resuspended in FACs buffer.

### Peripheral Blood Harvest

Six weeks after injection, blood was collected from all mice. Each blood sample was diluted (1:1) with PBS then layered on top of 500 ul of Ficoll Paque. The cells were spun at 800 rcf for 25 min at room temperature and the white blood cells at the interface between the Ficoll and aqueous layers were collected. These cells were washed, then resuspended in 0.5 ml FACs buffer.

### FACS Analysis

Two to 5 million cells were centrifuged at 400 rcf for 4 minutes at room temperature. The supernatant was discarded and the cells were resuspended in 200 μL of L/D NIR (1:200 dilution) and FC block (1:50 dilution). Cells were then stained with anti-human CD45 and anti-mouse CD45 antibodies. The proportion of human vs mouse CD45 cells were then analyzed using an Attune Flow cytometer.

### Results

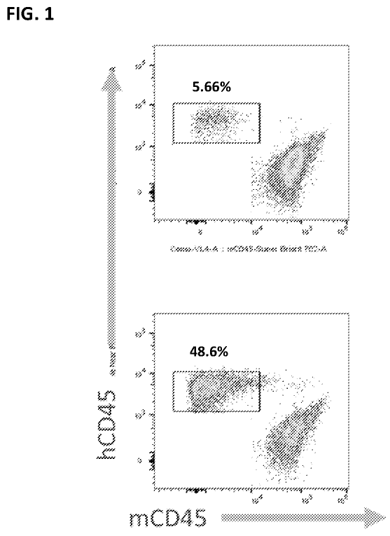

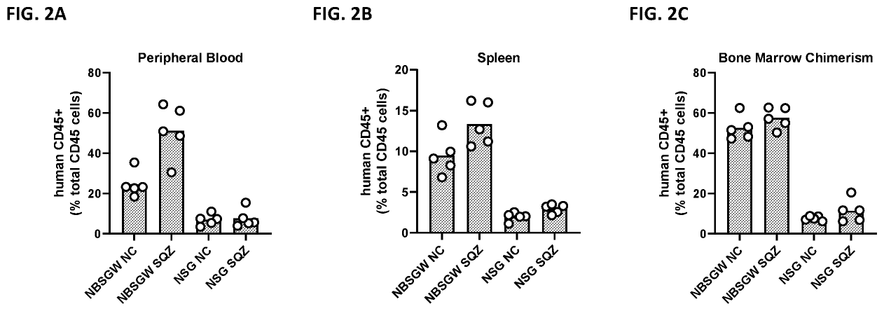

As shown in FIGS. 1 and 2C, the squeeze processed HSCs showed no detrimental effects to their engraftment potential. Significant percentage of the adoptively transferred squeeze processed human HSCs was observed in the bone marrow of both the NSG and NBSGW mice. Similar results were observed in the peripheral blood (FIG. 2A) and the spleen (FIG. 2B).

These results demonstrate that the squeeze processing methods provided herein do not have any negative effects on the engraftment potential of HSCs.

### Example 2: Analysis of CXCR4 and Bcl-2 Overexpression after Squeeze Processing

To assess whether the expression of certain payloads described herein can help promote HSC engraftment, CD34+ HSCs were loaded with mRNA encoding CXCR4 variants or Bcl-2 using the squeeze processing methods provided herein. The specific methods used are provided below.

### Squeeze Processing

One 5 M cell vial was thawed and diluted with FACs buffer. Cells were centrifuged at 400 rcf at room temperature then washed and resuspended to 2 M/ml in complete CD34+ media (StemSpan™ SFEM II containing 100 ng/ml SCF, TPO, Flt3-L). Cells were transferred to a 12 well non-treated plate and cultured at 37° C. In order to expand the HSCs, cells were cultured in complete CD34+ media for 4 days.

The cells were prepared for squeeze-processing. The cells were counted, washed and the concentration was adjusted to 10 M/mL by adding OptiMem. The following buffer solutions were prepared for each sample as shown in Table 6:

TABLE 6
Squeeze Processing Cell Suspension Solutions

| | |
|---|---|
| Group A | 100 μL OptiMem + 100 μL 2X HSCs |
| Group B | 100 μL OptiMEM + 100 μL 2X HSCs |
| Group C | 100 μL mRNA mix (109 μLRPMI + 17.9 μL |
| | CXCR4 mRNA + .55 μLNaCl) + 100 μL 2X |
| | HSCs |
| Group D | 100 μL mRNA mix (109 μLRPMI + 17.9 μL |
| | CXCR4 A175F mRNA + .55 μLNaCl) + 100 |
| | μL 2X HSCs |
| Group E | 100 μL mRNA mix (109 μLRPMI + 17.9 μL |
| | CXCR4 R334X + A175F mRNA + .55 |
| | μLNaCl) + 100 μL 2X HSCs |
| Group F | 100 μL mRNA mix (109 μLRPMI + 17.9 μL |
| | CXCR4 R334X mRNA + .55 μLNaCl) + 100 |
| | μL 2X HSCs |
| Group G | 100 μL mRNA mix (109 μLRPMI + 17.9 μL |
| | BCL-2 mRNA + .55 μLNaCl) + 100 μL 2X |
| | HSCs |
| Group H | 100 μL mRNA mix (109 μLRPMI + 17.9 μL |
| | BCL2 G101V mRNA + .55 μLNaCl) + 100 |
| | μL 2X HSCs |

As shown above in Table 6, Group A was used as a control (i.e., not contacted with any payload and not squeeze processed). Group B was squeeze processed without any payload at room temperature using a microfluidic constriction (10 μm depth, 3.5 μm width, and 70 μm length) at 30 psi. Groups C-H were squeeze processed at room temperature with one of the payloads shown above using, i.e., Group C: wild-type CXCR4 mRNA; Group D: CXCR4-A175F variant mRNA; Group E: CXCR4-R334X+A175F variant mRNA; Group F: CXCR4-R334X variant mRNA; Group G: wild-type Bcl-2 mRNA; and Group H: Bcl-2-G101V variant mRNA. Groups C-H were squeeze processed using the same conditions as in Group B (i.e., microfluidic constriction: 10 μm depth, 3.5 μm width, and 70 μm length; at 30 psi).

Afterwards, the cells from each of Groups A-H were separately transferred into their own 5 ml conical containing 2 mL CD34+ media. Cells from all groups were centrifuged separately, at 500 rcf for 5 minutes at room temperature. The supernatant was aspirated and the cell populations were separately resuspended in 1 mL of CD34+ media and cultured for 72 hours at 37° C.

### FACS Analysis

At 4, 24, 48 and 72 hrs, 200 ul of cells were aspirated and centrifuged at 400 rcf for 4 minutes at room temperature. The supernatant was discarded and the cells were resuspended in 50 μL of L/D aqua (1:200 dilution in FACs buffer) and FC block (1:50 dilution in FACs buffer). Cells were incubated at room temperature in the dark for 20 minutes, The cells were spun down at room temperature at 400 rcf for 4 minutes at room temperature. The cells were then incubated at 4C for 30 minutes with fluorescent anti-human CXCR4 antibody. After incubation 200 μL of FACs isolation buffer were added to each sample and the samples were spun at 400 rcf for 4 minutes. The cells were then fixed with paraformaldehyde for 20 minutes followed by staining with fluorescent anti-human BCL2 antibody for 30 min at room temperature. The supernatant was discarded and the cells resuspended with 200 μL of FACs isolation buffer. The cells were then analyzed using an Attune Flow cytometer.

### Results

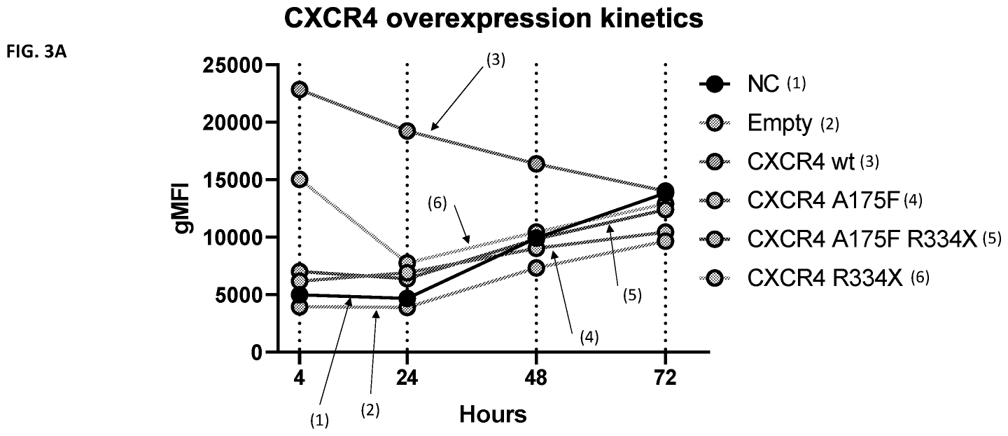

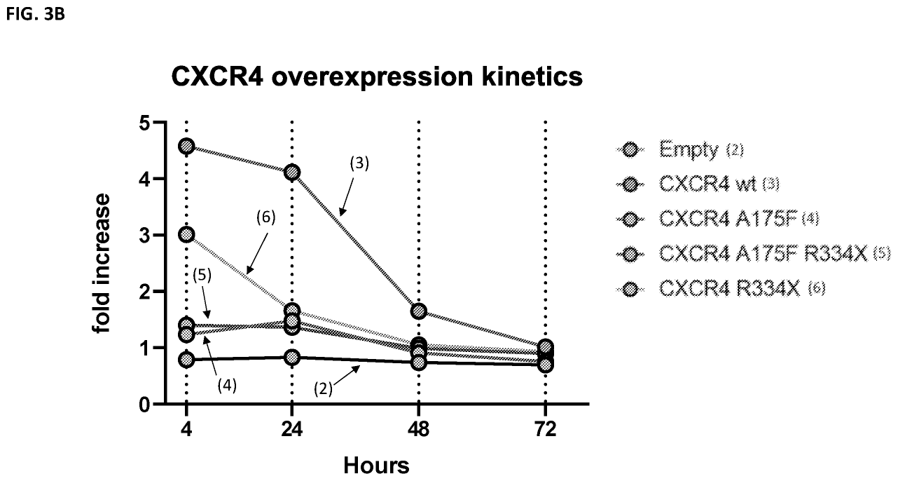

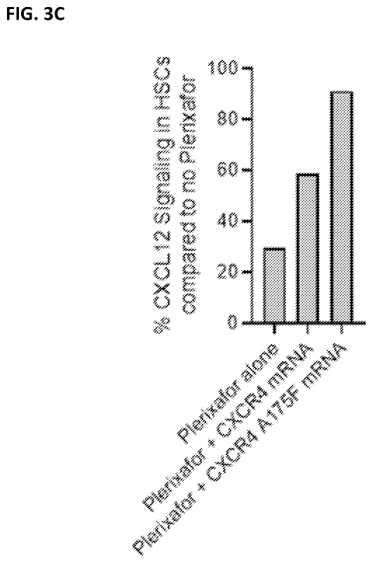

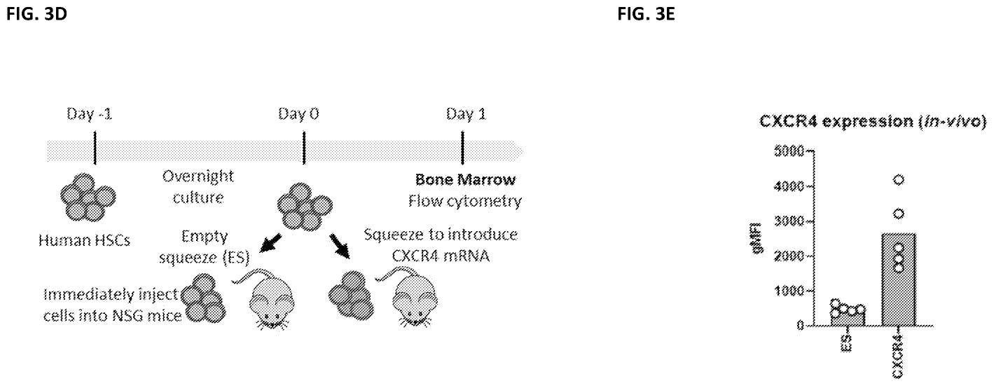

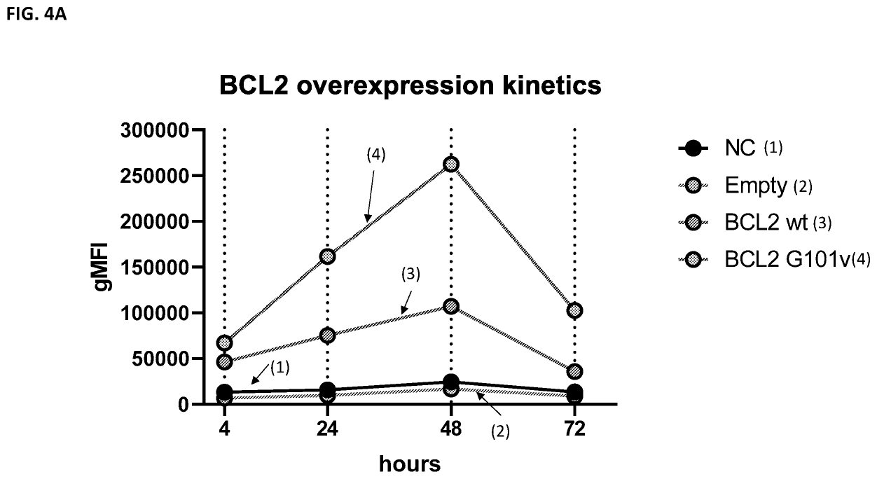

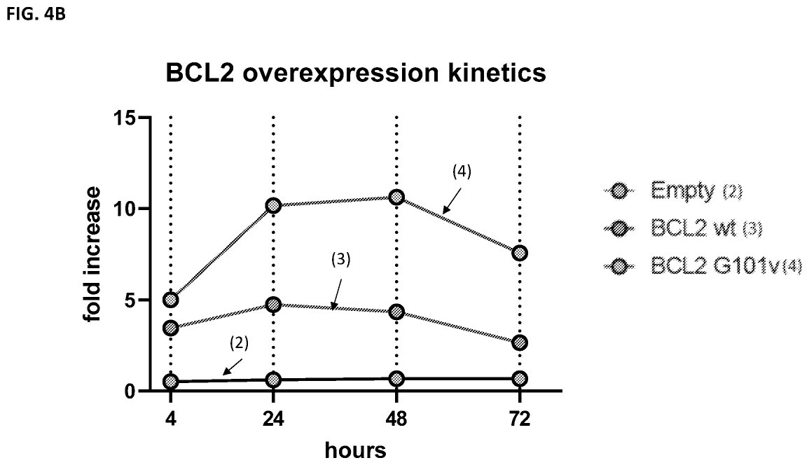

As shown in FIGS. 3A, 3B, 4A, and 4B, squeeze processing induced high expression of the delivered mRNA. All CXCR4 variants showed expression above background for at least 48 hours as measured in vitro (FIGS. 3A and 3B). Additionally, as shown in FIG. 3C, CD34+ HSCs squeeze processed with the CXCR4-A175F variant exhibited increased resistance to plerixafor (i.e., mobilization factor) (evidenced by CXCL12 signaling) as compared to the corresponding CD34+ HSCs that were squeeze processed with the wild-type CXCR4 mRNA. Not to be bound by any one theory, such result suggests that the modified HSCs would be more resistant to the effects of the mobilization factors (which promote the mobilization of cells from the bone marrow to the peripheral blood) and thereby, allow for improved engraftment within the bone marrow. Lastly, all BCL2 variants also showed expression above background for at least 72 hours (FIGS. 4A and 4B).

Collectively, the results provided herein demonstrate that the squeeze processing methods provided herein can be used to produce HSCs with enhanced engraftment capabilities.

### Example 3: In Vivo Analysis of CXCR4 Expression in Squeeze Processed CD34+ HSCs

To further assess whether modifying the HSCs to express certain homing receptors can allow for improved engraftment within the bone marrow, human CD34+ HSCs were cultured overnight and prepared for squeeze processing as generally described in Example 1. As shown in FIG. 3D, the CD34+ HSCs were squeeze processed with either no payload (squeeze processing alone) or a mRNA encoding the wild-type CXCR4, and then, adoptively transferred into NSG mice. Approximately 24 hours later, the mice were sacrified and CXCR4 expression was assessed within the bone marrow.

As shown in FIG. 3E, there was significantly higher CXCR4 expression observed in the bone marrow of animals that received the HSCs squeeze processed with the CXCR4 mRNA as compared to those animals that received the HSCs squeezed processed with no mRNA. These results confirm the in vivo expression of CXCR4 in HSCs squeeze processed with the CXCR4 mRNA after the adoptive transfer. Such results further demonstrate that the squeeze processing methods provided herein can be used to modify HSCs to express certain homing receptors (e.g., CXCR4), such that the modified HSCs exhibit improved homing to the bone marrow, which could allow for improved engraftment.

### Example 4: In Vivo Analysis of Bcl-2 Expression in Squeeze Processed CD34+ HSCs

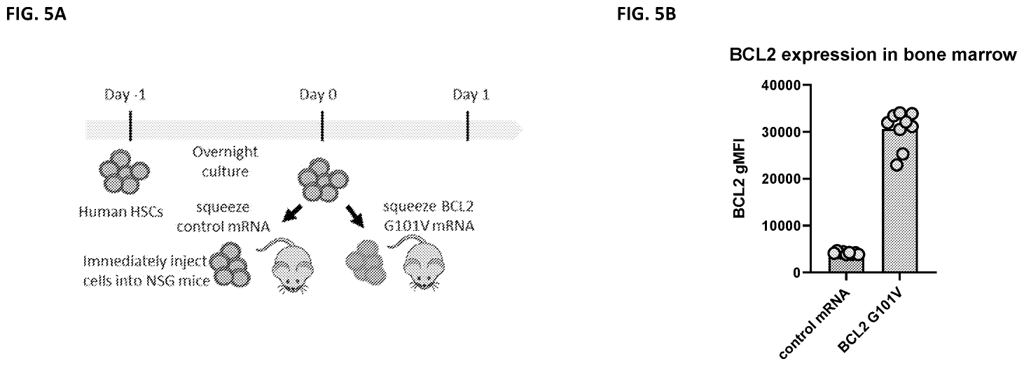

To further assess the effect that the survival factors described herein have on HSC engrafment, human CD34+ HSCs were cultured overnight and prepared for squeeze processing as generally described in Example 1. As shown in FIG. 5A, the CD34+ HSCs were squeeze processed with either a control mRNA or a mRNA encoding a Bcl-2 variant (i.e., G101V), and then, adoptively transferred into NSG mice. Approximately 24 hours later, the mice were sacrified and engraftment of the transferred HSCs was assessed within the bone marrow by measuring Bcl-2 expression using flow cytometry.

As shown in FIG. 5B, in mice that received the HSCs squeeze processed with the control mRNA, there was minimal Bcl-2 expression within the bone marrow. In contrast, significant Bcl-2 expression was observed within the bone marrow of mice that received the CD34+ HSCs modified to comprise the Bcl-2 variant mRNA, confirming that these cells are able to express Bcl-2 in vivo.

These results further demonstrate that the survival factors described herein (e.g., Bcl-2 G101V) can be useful in enhancing the engraftment of HSCs, for instance, when the survival factors are delivered to the HSCs using the squeeze processing methods described herein.

### Example 5: Analysis of the Resistance of CD34+ HSCs Comprising Bcl-2 Variant mRNA to Apoptotic Factors

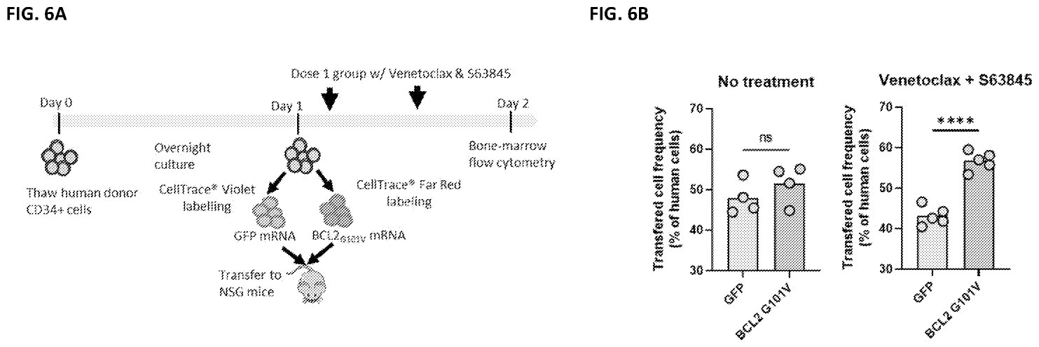

As described herein, the modified HSCs of the present disclosure (e.g., squeeze processed to comprise a survival factor, e.g., Bcl-2 variant) exhibit increased resistance to various apoptotic factors. To further demonstrate such improved property, human CD34+ HSCs were cultured overnight and prepared for squeeze processing as generally described in Example 1. As shown in FIG. 6A, the CD34+ HSCs were split and labeled with different fluorescent markers (either CELLTRACER Violet or CELLTRACER Far Red). Then, the cells labeled with the Violet fluorescent marker were squeeze processed with GFP mRNA, and the cells labeled with the Far Red fluorescent marker were squeeze processed with a mRNA encoding a Bcl-2 variant (i.e., G101V). Cells from each of the groups were mixed 1:1 and then adoptively transferred into NSG mice. Some of the mice were also dosed with Bcl-2 and MCL-1 inhibitors (i.e., venetoclax and S63845, respectively) (i.e., inhibitors of the anti-apoptotic factors). Approximately 24 hours after transfer, the mice were sacrificed and engraftment of the transferred cells within the bone marrow was assessed.

As shown in FIG. 6B, in the bone marrow of mice that were dosed with the apoptotic factors, there was a significantly greater frequency of the transferred HSCs modified to comprise the Bcl-2 variant mRNA, as compared to the frequency of the transferred HSCs modified to comprise the GFP mRNA.

These results confirm the enhanced ability of the modified HSCs of the present disclosure to resist the effects of anti-apoptotic inhibitors. Additionally, not to be bound by any one theory, the results further suggest that the combination of anti-apoptotic inhibitor and the modified HSCs described herein could be particularly useful in enhancing engraftment by selectively depleting the non-modified endogenous HSCs from the bone marrow, and allowing improved engraftment of the transferred modified HSCs.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

1. A method of producing a modified hematopoietic stem cell (HSC), comprising passing a cell suspension, which comprises a population of HSC, through a constriction under one or more parameters,
wherein passing the cell suspension through the constriction under the one or more parameters allows a payload to enter the HSC, and
wherein the payload is capable of modifying the HSC, such that the HSC exhibits:
(i) increased resistance to a mobilization factor, (ii) increased resistance to an apoptotic factor, (iii) increased resistance to a depleting factor, (iv) increased expression of a homing factor, or (v) a combination thereof.

2.-5. (canceled)

6. The method of claim 1, further comprising:
(i) increasing homing of a hematopoietic stem cell (HSC) to the bone marrow of a subject in need thereof when the HSC is administered to the subject; or
(ii) increasing survival of a hematopoietic stem cell in a subject in need thereof when the HSC is administered to the subject; or
(iii) promoting engraftment of a hematopoietic stem cell (HSC) in the bone marrow of a subject in need thereof when the HSC is administered to the subject.

7.-17. (canceled)

18. A method of producing a modified hematopoietic stem cell (HSC), comprising intracellularly delivering a payload into a HSC, wherein the payload is capable of modifying the HSC, such that the HSC exhibits: (i) increased resistance to a mobilization factor, (ii) increased resistance to an apoptotic factor, (iii) increased resistance to a depleting factor, (iv) increased expression of a homing factor, or (v) a combination thereof.

19.-22. (canceled)

23. The method of any claim 1, wherein the payload comprises a homing receptor, a cytokine, a growth factor, a cell adhesion molecule, a proliferative agent, a survival factor, a combination thereof, or a regulator thereof.

24. The method of claim 23, wherein:
(i) the homing receptor comprises a CXCR4, CXCR2, or both;
(ii) the cytokine comprises a stem cell factor (SCF), a Fms-related tyrosine kinase 3 ligand (Flt3L), or both;
(iii) the growth factor comprises a thrombopoietin (TPO);
(iv) the cell adhesion molecule comprises an integrin, a selectin, or both;
(v) the proliferative agent comprises an activator of a signaling pathway involved in cell proliferation;
(vi) the survival factor comprises a Bcl-2, a Bcl-xL, a MCL-1, a Ced-9, a bfl-1, or a combination thereof;
(vi) the regulator is capable of increasing the expression and/or activity of the homing receptor, the cytokine, the growth factor, the cell adhesion molecule, the proliferative agent, the survival factor, or a combination thereof; or
(vii) the regulator is capable of reducing or preventing (e.g., knocking down) the activity of an inhibitor of the homing receptor, the cytokine, the growth factor, the cell adhesion molecule, the proliferative agent, the survival factor, or a combination thereof.

25. The method of claim 24, wherein:
(i) the CXCR4 comprises an amino acid sequence which differs from the corresponding wild-type amino acid sequence set forth in SEQ ID NO: 1 or wherein the amino acid sequence of the CXCR4 comprises one of the following mutations: R334X, A175F, H113A, D171N, D262N, 1284A, H281A, Q200W, Q200A, or a combination thereof or
(ii) the Bcl-2 comprises an amino acid sequence which differs from the corresponding wild-type amino acid sequence set forth in SEQ ID NO: 9 or the amino acid sequence of the Bcl-2 comprises the G101V mutation, the D103Y mutation, or both.

26.-39. (canceled)

40. The method of claim 1, wherein the mobilization factor comprises a plerixaflor, an AMD3465, a GROβ, a G-CSF, an anti-CD117 antibody, or a combination thereof and/or wherein the apoptotic factor comprises a Bcl-2 inhibitor (e.g., Venetoclax), a MCL-1 inhibitor (e.g., S63845 or S64315), a BCL-XL inhibitor, or a combination thereof.

41. (canceled)

42. The method of claim 1, wherein the payload comprises a nucleic acid, and wherein the nucleic acid comprises a DNA, a RNA, or both, and wherein the RNA comprises a mRNA, a siRNA, a miRNA, a IncRNA, a tRNA, a shRNA, a self-amplifying mRNA (saRNA), a PNA, a locked nucleic acid (LNA), or a combination thereof.

43.-44. (canceled)

45. The method of claim 1, comprising contacting the HSC with the payload: (i) prior to the passing of the cell suspension through the constriction, (ii) during the passing of the cell suspension through the constriction, (iii) after the passing of the cell suspension through the constriction, or (iv) a combination thereof.

46.-50. (canceled)

51. The method of claim 1, wherein the one or more parameters are selected from a cell density; pressure; length, width, and/or depth of the constriction; diameter of the constriction; diameter of the cells;
temperature; entrance angle of the constriction; exit angle of the constriction; length, width, and/or width of an approach region; surface property of the constriction (e.g., roughness, chemical modification, hydrophilic, hydrophobic); operating flow speed; payload concentration; viscosity, osmolarity, salt concentration, serum content, and/or pH of the cell suspension; time in the constriction; shear rate in the constriction; type of payload, or a combination thereof.

52. The method of claim 51, wherein:
(i) the cell density is at least about 1×10⁵ cells/mL, at least about 2×10⁵ cells/mL, at least about 3×10⁵ cells/mL, at least about 4×10⁵ cells/mL, at least about 5×10⁵ cells/mL, at least about 6×10⁵ cells/mL, at least about 7×10⁵ cells/mL, at least about 8×10⁵ cells/mL, at least about 9×10⁵ cells/mL, at least about 1×10⁶ cells/mL, at least about 2×10⁶ cells/mL, at least about 3×10⁶ cells/mL, at least about 4×10⁶ cells/mL, at least about 5×10⁶ cells/mL, at least about 6×10⁶ cells/mL, at least about 7×10⁶ cells/mL, at least about 8×10⁶ cells/mL, at least about 9×10⁶ cells/mL, at least about 1×10⁷ cells/mL, at least about 2×10⁷ cells/mL, at least about 3×10⁷ cells/mL, at least about 4×10⁷ cells/mL, at least about 5×10⁷ cells/mL, at least about 6×10⁷ cells/mL, at least about 7×10⁷ cells/mL, at least about 8×10⁷ cells/mL, at least about 9×10⁷ cells/mL, at least about 1×10⁸ cells/mL, at least about 1.1×10⁸ cells/mL, at least about 1.2×10⁸ cells/mL, at least about 1.3×10⁸ cells/mL, at least about 1.4×10⁸ cells/mL, at least about 1.5×10⁸ cells/mL, at least about 2.0×10⁸ cells/mL, at least about 3.0×10⁸ cells/mL, at least about 4.0×10⁸ cells/mL, at least about 5.0×10⁸ cells/mL, at least about 6.0×10⁸ cells/mL, at least about 7.0×10⁸ cells/mL, at least about 8.0×10⁸ cells/mL, at least about 9.0×10⁸ cells/mL, or at least about 1.0×10⁹ cells/mL or more;
(ii) the pressure is at least about 20 psi, at least about 25 psi, at least about 30 psi, at least about 35 psi, at least about 40 psi, at least about 45 psi, at least about 50 psi, at least about 55 psi, at least about 60 psi, at least about 65 psi, at least about 70 psi, at least about 75 psi, at least about 80 psi, at least about 85 psi, at least about 90 psi, at least about 95 psi, at least about 100 psi, at least about 110 psi, at least about 120 psi, at least about 130 psi, at least about 140 psi, or at least about 150 psi;
(iii) the diameter of the constriction is about 20%, about 30%, about 40%, about 50%, about 60%, about 70%, about 80%, about 90%, or about 99% of the diameter of the HSC;
(iv) the length of the constriction is less than about 0.1 μm, less than about 0.2 μm, less than about 0.3 μm, less than about 0.4 μm, less than about 0.5 μm, less than about 0.6 μm, less than about 0.7 μm, less than about 0.8 μm, less than about 0.9 μm, less than about 1 μm, less than about 2.5 μm, less than about 5 μm, less than about 7.5 μm, less than about 10 μm, less than about 12.5 μm, less than about 15 μm, less than about 20 μm, less than about 30 μm, less than about 40 μm, less than about 50 μm, less than about 60 μm, less than about 70 μm, less than about 80 μm, less than about 90 μm, or less than about 100 μm;
(v) the width of the constriction is less than about 1 μm, less than about 2 μm, less than about 3 μm, less than about 4 μm, less than about 5 μm, less than about 6 μm, less than about 7 μm, less than about 8 μm, less than about 9 μm, or less than about 10 μm; and/or
(vi) the depth of the constriction is at least about 2 μm, at least about 3 μm, at least about 4 μm, at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 110 μm, or at least about 120 μm.

53.-66. (canceled)

67. The method of claim 45, wherein the HSC is contacted with multiple payloads (i) prior to the passing of the cell suspension through the constriction, (ii) during the passing of the cell suspension through the constriction, (iii) after the passing of the cell suspension through the constriction, or (iv) a combination thereof, such that passing the cell suspension through the constriction under the one or more parameters allow at least two or more of the multiple payloads to enter the HSC.

68. (canceled)

69. The method of claim 67, wherein the multiple payloads enter the cell concurrently or sequentially.

70. (canceled)

71. The method of claim 1, comprising passing the cell suspension through a plurality of constrictions, wherein
(i) each constriction of the plurality of constrictions are the same or wherein one or more of the constrictions of the plurality of constrictions are different; or
(ii) each constriction of the plurality of constrictions is associated with the same payload or wherein one or more of the plurality of constrictions is associated with a different payload.

72.-82. (canceled)

83. The method of claim 71, wherein the plurality of constrictions comprise a first constriction associated with a first payload and a second constriction associated with a second payload, wherein the cell suspension is passed through the first constriction allowing the first payload to enter the HSC, and then the cell suspension is passed through the second constriction allowing the second payload to enter the HSC.

84.-86. (canceled)

87. A composition comprising a population of modified hematopoietic stem cells (HSCs), wherein the modified HSCs comprise a payload, which is capable of: (i) increasing the resistance of the modified HSCs to a mobilization factor, (ii) increasing the resistance of the modified HSCs to an inhibitor of an anti-apoptotic factor, (iii) increasing the resistance of the modified HSCs to a depleting factor, (iv) increasing the expression of a homing factor on the modified HSCs, or (v) a combination thereof.

88.-92. (canceled)

93. The composition of claim 87, wherein the payload comprises a homing receptor, a cytokine, a growth factor, a cell adhesion molecule, a proliferative agent, a survival factor, a combination thereof, or a regulator thereof.

94. The composition of claim 93, wherein;
(i) the homing receptor comprises a CXCR4, CXCR2, or both;
(ii) the cytokine comprises a stem cell factor (SCF), a Fms-related tyrosine kinase 3 ligand (Flt3L), or both;
(iii) the growth factor comprises a thrombopoietin (TPO);
(iv) the cell adhesion molecule comprises an integrin, a selectin, or both;
(v) the proliferative agent comprises an activator of a signaling pathway involved in cell proliferation;
(vi) the survival factor comprises a Bcl-2, a Bcl-XL, a MCL-1, a Ced-9, a bfl-1, or a combination thereof;
(vi) the regulator is capable of increasing the expression and/or activity of the homing receptor, the cytokine, the growth factor, the cell adhesion molecule, the proliferative agent, the survival factor, or a combination thereof or
(vii) the regulator is capable of reducing or preventing (e.g., knocking down) the activity of an inhibitor of the homing receptor, the cytokine, the growth factor, the cell adhesion molecule, the proliferative agent, the survival factor, or a combination thereof.

95. The composition of claim 94, wherein:
(i) the CXCR4 comprises an amino acid sequence which differs from the corresponding wild-type amino acid sequence set forth in SEQ ID NO: 1 or wherein the amino acid sequence of the CXCR4 comprises one of the following mutations: R334X, A175F, H113A, D171N, D262N, 1284A, H281A, Q200W, Q200A, or a combination thereof or
(ii) the Bcl-2 comprises an amino acid sequence which differs from the corresponding wild-type amino acid sequence set forth in SEQ ID NO: 9 or wherein the amino acid sequence of the Bcl-2 comprises the G101V mutation, the D103Y mutation, or both.

96.-115. (canceled)

116. The composition of claim 87 for use in a method of treating a disease or disorder in a subject in need thereof, wherein the method comprises administering the composition to the subject and wherein the disease or disorder comprises a blood disorder, an immune disorder or a metabolic disorder.

117.-126. (canceled)


SEQUENCE LISTING (12 sequences)

SEQ ID NO: 1 - protein, 352 aa, Homo sapiens
  source 1..352: note = CXCR4
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - protein, 352 aa, synthetic construct
  source 1..352: note = CXCR4 (A175F) Variant
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFFNVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - protein, 333 aa, synthetic construct
  source 1..333: note = CXCR4 (R334X) Variant
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGK

SEQ ID NO: 4 - protein, 333 aa, synthetic construct
  source 1..333: note = CXCR4 (A175F+R334X) Variant
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFFNVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGK

SEQ ID NO: 5 - DNA, 1056 bp, Homo sapiens
  source 1..1056: mol_type = genomic DNA; note = CXCR4
atggaaggcatcagcatctacaccagcgacaactacacagaggaaatgggcagcggcgactacgacagcatgaaagagccttgtttccgggaagagaacgccaatttcaacaagattttcctgccaacaatctatagcatcatctttctgaccggcatcgtgggaaatggcctggtgatcctggtcatgggctaccagaagaagctgagatctatgacagataagtaccggctgcacctgagcgtggctgatctgctgttcgtgatcaccctgcccttctgggccgtggacgccgtggccaactggtatttcggcaacttcctgtgcaaggccgtgcacgtgatctacaccgtgaacctgtacagctccgtgctgatcctggcttttatcagcctggatagatacctggccatcgtgcacgccaccaactcacagagacctagaaagctgctggccgagaaggtggtctacgtgggagtgtggatccctgccctgctgctcaccatccccgacttcatcttcgccaacgtgtccgaggccgacgaccggtacatctgcgacagattctaccccaacgacctgtgggtcgtggtgttccagttccaacacatcatggtgggcctgattctgcctggcatcgtgatcctgagctgctactgcatcatcatcagcaagctgtcccacagcaaaggccaccagaaaagaaaggccctgaaaaccaccgtgatcctgatcctcgcttttttcgcgtgctggctgccttactacatcggcattagcatcgatagcttcatcctgctggaaatcatcaagcagggttgtgaattcgagaacaccgtgcataagtggatctctattacagaggccctggctttcttccactgctgtctgaatcctatcctgtacgcctttctgggagcaaagttcaagaccagcgcccagcacgccctgacctctgtttctcggggaagcagcctgaagatcctctccaagggcaagagaggcggccacagcagcgtgtcgacagagagcgagagcagctcttttcattcttct

SEQ ID NO: 6 - DNA, 1059 bp, synthetic construct
  source 1..1059: mol_type = other DNA; note = CXCR4 (A175F) Variant
atggaaggcatcagcatctatacatccgacaactacacagaggaaatgggatctggcgactacgacagcatgaaagagccttgtttccgggaagagaatgccaacttcaacaagatcttcttgcctacaatctactccatcatctttctgaccggcatcgtgggaaacggcctggtgatcctcgtgatgggctaccagaaaaagcttagaagcatgaccgataagtacagactgcacctgagcgtcgccgatctgctgttcgtgatcacactgccattctgggccgtggacgccgtggccaattggtacttcggcaacttcctgtgcaaggccgtgcatgtgatctatacagtgaacctgtacagcagcgtcctgatcctggcttttatctctctggatcggtacctggctatcgtgcacgccaccaacagccagagacctagaaagctgctggctgagaaggtggtgtacgtgggagtgtggatccccgccctgctgctgaccatccctgatttcatcttcttcaatgtgtctgaggccgacgacagatacatctgcgacaggttctaccccaacgacctgtgggtggtcgtgttccaatttcagcacatcatggtgggcctgatcctgcctggcatcgttattctgagctgttactgcatcattatctccaagctgtctcacagcaagggacaccagaagcggaaggccctcaaaaccaccgtgatcctgatcctggccttcttcgcctgctggctgccttactacatcggcatcagcatcgacagctttatcctgctggaaatcattaagcagggctgtgaattcgagaacaccgtgcacaagtggatcagcatcaccgaggctctggctttcttccactgctgcctgaaccccatcctgtacgcctttctgggcgccaagttcaagaccagcgcccagcacgccctgacaagcgtgtccagaggctctagcctgaaaatcctgtccaagggcaagagaggcggccacagcagcgtgagcaccgagagcgagagctctagcttccactccagctga

SEQ ID NO: 7 - DNA, 1001 bp, synthetic construct
  source 1..1001: mol_type = other DNA; note = CXCR4 (R334X) Variant
atggaaggcatcagcatctacaccagcgacaactacacagaggaaatgggcagcggcgactacgacagcatgaaagagccttgtttccgggaagagaacgccaatttcaacaagattttcctgccaacaatctatagcatcatctttctgaccggcatcgtgggaaatggcctggtgatcctggtcatgggctaccagaagaagctgagatctatgacagataagtaccggctgcacctgagcgtggctgatctgctgttcgtgatcaccctgcccttctgggccgtggacgccgtggccaactggtatttcggcaacttcctgtgcaaggccgtgcacgtgatctacaccgtgaacctgtacagctccgtgctgatcctggcttttatcagcctggatagatacctggccatcgtgcacgccaccaactcacagagacctagaaagctgctggccgagaaggtggtctacgtgggagtgtggatccctgccctgctgctcaccatccccgacttcatcttcgccaacgtgtccgaggccgacgaccggtacatctgcgacagattctaccccaacgacctgtgggtcgtggtgttccagttccaacacatcatggtgggcctgattctgcctggcatcgtgatcctgagctgctactgcatcatcatcagcaagctgtcccacagcaaaggccaccagaaaagaaaggccctgaaaaccaccgtgatcctgatcctcgcttttttcgcgtgctggctgccttactacatcggcattagcatcgatagcttcatcctgctggaaatcatcaagcagggttgtgaattcgagaacaccgtgcataagtggatctctattacagaggccctggctttcttccactgctgtctgaatcctatcctgtacgcctttctgggagcaaagttcaagaccagcgcccagcacgccctgacctctgtttctcggggaagcagcctgaagatcctctccaagggcaatga

SEQ ID NO: 8 - DNA, 1002 bp, synthetic construct
  source 1..1002: mol_type = other DNA; note = CXCR4 (A175F+R334X) Variant
atggaaggcatctccatctacacctctgataactacacagaggagatgggcagcggcgactacgacagcatgaaggaaccttgtttccgggaagagaacgccaatttcaacaagatcttcctgcctacaatctacagcatcatcttcctcacaggcatcgtgggcaacggcctggtgatcctggtgatgggatatcagaaaaagcttagatccatgaccgataagtacagactgcacctgagcgtggctgatctgctgttcgtgatcaccctgccattctgggccgtggacgctgtggccaactggtacttcggcaatttcctgtgcaaggccgtgcacgtgatctacaccgtaaacctgtacagcagcgtgctgatcctggcttttatctctctggatagatacctggctatcgtgcatgccaccaacagccagaggcctagaaagctgctggccgagaaggtggtgtacgtgggtgtttggatccctgccttgctgctgacaatccccgacttcatcttcttcaatgtgtctgaggccgacgacagatatatctgcgaccggttctaccccaacgacctgtgggtcgtggtgttccaatttcagcacatcatggtcggactgatcctgcctggcatcgtgatcctgagctgctactgcatcattatttctaaactgagccacagcaagggccaccagaagcggaaagccctgaagaccaccgtgatcctgatcctggcttttttcgcctgttggctgccctactacatcggcatcagcatcgactccttcatcctgctggaaatcattaagcagggctgtgaattcgagaacaccgtgcacaagtggatcagcattaccgaggccctggccttctttcactgctgcctgaaccctatcctgtacgcctttctgggcgccaagttcaagaccagcgcccagcacgccctgacaagcgtgtccagaggatcttctctgaagatcctgagcaagggcaaatga

SEQ ID NO: 9 - protein, 239 aa, Homo sapiens
  source 1..239: note = Bcl-2
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 10 - protein, 239 aa, Homo sapiens
  source 1..239: note = Bcl-2 (G101V) Variant
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAVDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 11 - DNA, 720 bp, Homo sapiens
  source 1..720: mol_type = genomic DNA; note = Bcl-2
atggctcacgccggcagaaccggctacgacaacagagaaatcgtgatgaagtacatccactacaagctgagccagcggggctacgaatgggatgctggcgacgttggagctgctcctcctggtgctgctcctgctcctggaatctttagcagccagcctggccacacacctcatcctgccgcttctagagatcccgtggccagaacaagccctctgcagacaccagctgctccaggtgctgcagctggacctgcactttctccagtgcctccagtggtgcacctgacactgagacaggccggcgacgacttcagcagaagatacagacgggacttcgccgagatgtccagccagctgcatctgacccctttcacagccagaggcagattcgccaccgtggtggaagaactgttccgcgacggtgtcaactggggcagaatcgtggccttcttcgagttcggcggcgtgatgtgtgtggaaagcgtgaaccgggaaatgagccctctggtggacaatatcgccctgtggatgaccgagtacctgaaccggcatctgcacacatggatccaggacaacggcggctgggatgccttcgtggaactgtacggacctagcatgaggcccctgttcgacttctcttggctgagcctgaaaaccctgctgtctctggcccttgtgggcgcctgtattacactgggagcctacctgggccacaaatga

SEQ ID NO: 12 - DNA, 720 bp, Homo sapiens
  source 1..720: mol_type = genomic DNA; note = Bcl-2 (G101V) Variant
atggctcacgccggcagaaccggctacgacaacagagaaatcgtgatgaagtacatccactacaagctgagccagcggggctacgaatgggatgctggcgacgttggagctgctcctcctggtgctgctcctgctcctggaatctttagcagccagcctggccacacacctcatcctgccgcttctagagatcccgtggccagaacaagccctctgcagacaccagctgctccaggtgctgcagctggacctgcactttctccagtgcctccagtggtgcacctgacactgagacaggccgtggacgacttcagccggcggtatagaagagacttcgccgagatgtccagccagctgcatctgacccctttcacagccagaggcagattcgccaccgtggtggaagaactgttccgcgacggtgtcaactggggcagaatcgtggccttcttcgagttcggcggcgtgatgtgtgtggaaagcgtgaaccgggaaatgagccctctggtggacaatatcgccctgtggatgaccgagtacctgaaccggcatctgcacacatggatccaggacaacggcggctgggatgccttcgtggaactgtacggacctagcatgaggcccctgttcgacttctcttggctgagcctgaaaaccctgctgtctctggcccttgtgggcgcctgtattacactgggagcctacctgggccacaaatga